(19) United States
(12) Patent Application Publication
(10) Pub. No.: US 2026/0109758 A1
(43) Pub. Date: Apr. 23, 2026

(54) RECOMBINANT BISPECIFIC ANTIBODIES TARGETING TSLP AND IL4R

(71) Applicant: BIOSION, INC., Nanjing, Jiangsu (CN)

(72) Inventors: Mingjiu CHEN, Hockessin, DE (US); Mark Zhiqing MA, Nanjing (CN); Zeyu PENG, Nanjing (CN)

(21) Appl. No.: 19/114,283

(22) PCT Filed: Sep. 20, 2023
(86) PCT No.: PCT/CN2023/120089

§ 371 Date: Mar. 21, 2025

Related U.S. Application Data
(60) Provisional application No. 63/376,626, filed on Sep. 22, 2022.

(51) Int. Cl. C07K 16/24; A61P 11/06; A61P 17/00; C07K 16/28

(52) U.S. Cl. CPC C07K 16/244; A61P 11/06; A61P 17/00; C07K 16/2866; C07K 2317/24; C07K 2317/31; C07K 2317/622; C07K 2317/76; C07K 2317/92

(57) ABSTRACT

The present disclosure provides a recombinant bispecific antibody comprising i) a TSLP binding domain, and ii) an IL4R binding domain. The present disclosure provides a nucleic acid molecule encoding the recombinant bispecific antibody, a vector comprising the nucleic acid and a host cell transformed or transfected with the vector or with such a nucleic acid molecule. Furthermore, the present disclosure provides a process for the production of the recombinant bispecific antibody, a medical use of the recombinant bispecific antibody and a kit comprising the recombinant bispecific antibody.

### FIELD OF THE INVENTION

The present disclosure relates generally to a recombinant bispecific antibody, which is able to bind TSLP and IL4R, with high affinity and functionality. A nucleic acid molecule encoding the recombinant bispecific antibody, an expression vector, a host cell and a method for expressing the recombinant bispecific antibody are also provided. The present disclosure further provides a pharmaceutical composition which may comprise the recombinant bispecific antibody, as well as a treatment method using the pharmaceutical composition of the disclosure.

### BACKGROUND OF THE INVENTION

### TSLP

Thymic stromal lymphopoietin (TSLP) is an epithelial cell-derived cytokine. It closely relates to IL-7, with which it shares an overlapping but distinct biological profile. TSLP binds to a heterodimeric receptor complex composed of the TSLP receptor chain (TSLPR, also known as CRLF2) and the IL-7 receptor alpha chain (IL-7Rα). TSLP mRNAs are expressed predominantly by epithelial cells in the thymus, lung, skin, intestine and tonsils, as well as stromal cells and mast cells. While TSLPR mRNAs are found on many immune cell types, including dendritic cells (DCs), T cells, B cells, mast cells, NK cells and monocytes (Rui He et al., (2010) Annals of the New York Academy of Sciences 1183:13-24; Quentmeier H et al., (2001) Leukemia 15(8):1286-1292; Rimoldi M et al., (2005) Nature immunology 6(5):507-514).

The TSLP-triggered signaling pathways have been extensively studied. For example the TSLP protein may induce DC polarization to drive T helper (Th)2 cell differentiation and Th2 cytokine production during the induction phase of the immune response, and also may directly promote T cell expansion and amplify Th2 cytokine secretion. Accordingly, TSLP is believed to be a master regulator of Th2 driven inflammation, and upregulation of TSLP is linked to the pathogenesis of Th2-related diseases, such as atopic dermatitis, and asthma (Rui He et al., (2010) supra; Ito T et al., (2005) The Journal of Experimental Medicine 202(9):1213-1223; He R et al., (2008) Proc Natl Acad Sci USA 105(33):11875-11880). On the other hand, TSLP mediates several immune homeostatic functions in the gut and the thymus. For example, TSLP is upregulated in gut epithelial cell lines upon bacterial stimulation in a strain-dependent fashion, which synergizes with the transforming growth factor-beta to promote Treg cell differentiation. TSLP is also produced by primary human intestinal epithelial cells for the conditioning of CD103⁺ DCs to a tolerogenic phenotype (Katerina Tsilingiri et al., (2017) Cellular and Molecular Gastroenterology and Hepatology 3(2):174-182; Zeuthen L H et al., (2008) Immunology 123:197-208; Iliev I D et al., (2009) Gut 58:1481-1489).

The dual role of TSLP on the immune system leads to the discovery of two isoforms, a long isoform and a short isoform composed of the last 63 amino acid residues of the longer one. These two are controlled by different promoter regions, and are expressed depending on the context, tissue and stimulus (Harada M et al., (2011) American Journal of Respiratory Cell and Molecular Biology 44:787-793). The isoform expression pattern has also been investigated in some TSLP-related diseases. For example, over-expression of the long TSLP was observed in asthma, ulcerative colitis, atopic dermatitis and psoriasis, and reduced expression of the long TSLP was found in celiac disease. Furthermore, the expression of the short TSLP was downregulated in Crohn's disease, celiac disease and atopic dermatitis (Katerina Tsilingiri et al., (2017) supra; Fornasa G et al., (2015) J Allergy Clin Immunol 136:413-422).

### IL-4R

Interleukin-4 (IL-4) and IL-13 are two factors central to type 2 immunity, and required to drive most of the key hallmarks associated with type 2 inflammation, such as immunoglobulin E production, and innate cell recruitment to inflammation sites (Gruning G et al., (1998) Science 282:2261-2263; Rankin J A et al., (1996) Proc Natl Acad Sci USA 93:7821-7825; Wills-Karp M et al., (1998) Science 282:2258-2261).

The two cytokines bind cell surface receptors to regulate cellular functions and activate transcriptional machinery. In specific, IL-4 first binds to IL-4Rα chain with picomolar affinity, and recruits IL-2Rγc (γc) chain to form type I IL-4 receptor complex, or alternatively recruits IL-13Rα1 to form type II IL-4 receptor complex. The level or availability of IL-2Rγ and IL-13Rα1 determines which one to be recruited in receptor complex formation. The formation of type II IL-4 receptor complex may also be initiated with the binding of IL-13 to IL-13Rα1 chain with nanomolar affinity, resulting in further recruitment of the IL-4Rα chain.

Once the IL-4 receptor complexes are assembled, intracellular signaling molecules are activated, STAT6 (signal transducer and activator of transcription 6) and IRS (insulin receptor substrate) signaling for example are responsive to the type I IL-4 receptor activation (Heller N M et al., (2008) Sci Signal 1(51):ra17-ra17). The STAT6 signaling is important in TH2 cell differentiation and IL-4 production (Gadani S P et al., (2012) J Immunol 189:4213-4219) and the IRS molecules activate signaling pathways including PI3K and mTOR (Gadani S P et al., (2012) J Immunol 189:4213-4219).

Researches have suggested that excessive IL-4/IL-13 signaling might cause allergic diseases, and therefore several therapeutic antibodies have been developed to modify IL-4 and IL-13 mediated signaling, such as Leprikizumab, Anrukinzumab and Tralokinumab binding IL-13, and Pascolizumab targeting IL-4. Dupilumab is a fully human monoclonal antibody against IL-4Rα, which inhibits both IL-4 and IL-13 signaling and is approved for treating patients with type 2 inflammatory diseases, including AD (atopic dermatitis), asthma, and CRSwNP (chronic rhinosinusitis with nasal polyposis) (Haddad, E B. et al. (2022) Dermatol Ther 12:1501-1533). Moreover, a STAT6 inhibitor has been found to inhibit prostate cancer cell growth, suggesting that targeting IL-4/IL-13 may benefit cancer treatment (Nappo G et al., (2017) Oncogenesis 2017, 6(5): e342).

### Bispecific or Multi-Specific Antibodies

Given that some monospecific antibodies or proteins have limited therapeutic efficacy, bi- or tri-specific antibodies are being gradually developed in recent years and show promising effects in pre-clinical and clinical tests.

Citation or identification of any document in this application is not an admission that such document is available as prior art to the present invention.

### SUMMARY OF THE INVENTION

The inventors of the present application have designed and prepared a recombinant bispecific antibody that targets both TSLP and IL4R, and has i) comparable binding affinity/activity to TSLP and/or IL4R, ii) comparable blocking activity on IL4-IL4R binding/interaction and/or TSLP-TSLPR/IL7Rα binding/interaction, and/or iii) comparable, if not higher, blocking activity on TSLP and/or IL4 induced or mediated signaling, as compared to the prior art monospecific anti-IL4R or anti-TSLP antibodies, e.g., Dupilumab and Tezepelumab. In particular, the recombinant bispecific antibody of the disclosure shows significantly higher inhibitory effect on TSLP+IL4 induced signaling, including CCL-17 production by PBMCs, than the monospecific anti-IL4R or anti-TSLP antibodies, and even the combination of Dupilumab and Tezepelumab.

The recombinant bispecific antibody of the disclosure may be used for in vitro and in vivo assays and treatment of diseases associated with TSLP, IL4 and/or IL13 signaling such as inflammatory diseases.

In a first aspect, the disclosure provides a recombinant bispecific antibody that binds both TSLP and IL4R, which may comprise a TSLP binding domain and an IL4R binding domain.

The TSLP binding domain may be an anti-TSLP antibody, e.g., an IgG antibody, or an antigen-binding portion thereof such as a Fab, a Fv, or a single-chain variable region (scFv). In certain embodiments, the TSLP binding domain is antagonistic.

The IL4R binding domain may be an anti-IL4R antibody, e.g., an IgG antibody, or an antigen-binding portion thereof such as a Fab, a Fv, or a single-chain variable region (scFv). In certain embodiments, the IL4R binding domain is antagonistic.

The recombinant bispecific antibody of the disclosure may comprise:
i) an anti-TSLP antibody or an antigen binding portion thereof, which may comprise an anti-TSLP heavy chain variable region, an anti-TSLP light chain variable region, a heavy chain constant region, and a light chain constant region, and
ii) an anti-IL4R single-chain variable region (scFv).

The anti-IL4R scFv may be linked to the N-terminus of the anti-TSLP heavy chain variable region, the N-terminus of the anti-TSLP light chain variable region, the C-terminus of the heavy chain constant region, or the C-terminus of the light chain constant region.

The recombinant bispecific antibody of the disclosure may comprise:
i) an anti-IL4R antibody or an antigen binding portion thereof, which may comprise an anti-IL4R heavy chain variable region, an anti-IL4R light chain variable region, a heavy chain constant region, and a light chain constant region, and
ii) an anti-TSLP single-chain variable region (scFv).

The anti-TSLP scFv may be linked to the N-terminus of the anti-IL4R heavy chain variable region, the N-terminus of the anti-IL4R light chain variable region, the C-terminus of the heavy chain constant region, or the C-terminus of the light chain constant region.

In certain embodiments, the recombinant bispecific antibody of the disclosure may comprise:
i) an antagonistic anti-TSLP antibody, comprising an anti-TSLP heavy chain variable region, a heavy chain constant region, an anti-TSLP light chain variable region, and an light chain constant region, and
ii) an antagonistic anti-IL4R single-chain variable region (scFv), comprising an anti-IL4R heavy chain variable region and an anti-IL4R light chain variable region.

The anti-IL4R scFv may be linked to the N-terminus of the anti-TSLP heavy chain variable region, the N-terminus of the anti-TSLP light chain variable region, the C-terminus of the heavy chain constant region, or the C-terminus of the anti-TSLP light chain constant region.

Alternatively, the recombinant bispecific antibody of the disclosure may comprise:
i) an antagonistic anti-IL4R antibody, comprising an anti-IL4R heavy chain variable region, a heavy chain constant region, an anti-IL4R light chain variable region, and an light chain constant region, and
ii) an antagonistic anti-TSLP single-chain variable region (scFv), comprising an anti-TSLP heavy chain variable region and an anti-TSLP light chain variable region.

The anti-TSLP scFv may be linked to the N-terminus of the anti-IL4R heavy chain variable region, the N-terminus of the anti-IL4R light chain variable region, the C-terminus of the heavy chain constant region, or the C-terminus of the anti-IL4R light chain constant region.

The recombinant bispecific antibody of the disclosure, in certain embodiments, may comprise:
i) a first polypeptide chain and a second polypeptide chain each comprising an anti-IL4R heavy chain variable region, a heavy chain constant region, and an anti-TSLP scFv, and
ii) a third polypeptide chain and a fourth polypeptide chain each comprising an anti-IL4R light chain variable region and an light chain constant region;
i) a first polypeptide chain and a second polypeptide chain each comprising an anti-IL4R heavy chain variable region, and a heavy chain constant region, and
ii) a third polypeptide chain and a fourth polypeptide chain each comprising an anti-IL4R light chain variable region, an light chain constant region, and an anti-TSLP scFv;
i) a first polypeptide chain and a second polypeptide chain each comprising an anti-TSLP heavy chain variable region, a heavy chain constant region, and an anti-IL4R scFv, and
ii) a third polypeptide chain and a fourth polypeptide chain each comprising an anti-TSLP light chain variable region and an light chain constant region; or
i) a first polypeptide chain and a second polypeptide chain each comprising an anti-TSLP heavy chain variable region, and a heavy chain constant region, and
ii) a third polypeptide chain and a fourth polypeptide chain each comprising an anti-TSLP light chain variable region, an light chain constant region, and an anti-IL4R scFv,
wherein the anti-IL4R heavy chain variable region in the first polypeptide chain and the anti-IL4R light chain variable region in the third polypeptide chain associate to form an IL4R binding domain, the anti-IL4R heavy chain variable region in the second polypeptide chain and the anti-IL4R light chain variable region in the fourth polypeptide chain associate to form an IL4R binding domain,
wherein the anti-TSLP heavy chain variable region in the first polypeptide chain and the anti-TSLP light chain variable region in the third polypeptide chain associate to form a TSLP binding domain, the anti-TSLP heavy chain variable region in the second polypeptide chain and the anti-TSLP light chain variable region in the fourth polypeptide chain associate to form a TSLP binding domain, and
wherein the heavy chain constant region in the first polypeptide chain and the heavy chain constant region in the second polypeptide chain are associated together.

In certain embodiments, the first polypeptide chain and the second polypeptide chain each comprises, from N- to C-terminus, the anti-IL4R scFv, an optional linker, the anti-TSLP heavy chain variable region, and the heavy chain constant region, and the third polypeptide chain and the fourth polypeptide chain each comprises, from N- to C-terminus, the anti-TSLP light chain variable region and the light chain constant region, wherein the anti-IL4R scFv in the first and second polypeptide chains each comprises from N- to C-terminus an anti-IL4R heavy chain variable region, an optional linker and an anti-IL4R light chain variable region, or an anti-IL4R light chain variable region, an optional linker, and an anti-IL4R heavy chain variable region.

In certain embodiments, the first polypeptide chain and the second polypeptide chain each comprises, from N- to C-terminus, the anti-TSLP heavy chain variable region, and the heavy chain constant region, and the third polypeptide chain and the fourth polypeptide chain each comprises, from N- to C-terminus, the anti-IL4R scFv, an optional linker, the anti-TSLP light chain variable region and the light chain constant region, wherein the anti-IL4R scFv in the third and fourth polypeptide chains each comprises from N- to C-terminus an anti-IL4R heavy chain variable region, an optional linker, and an anti-IL4R light chain variable region, or an anti-IL4R light chain variable region, an optional linker, and an anti-IL4R heavy chain variable region.

In certain embodiments, the first polypeptide chain and the second polypeptide chain each comprises, from N- to C-terminus, the anti-TSLP heavy chain variable region, the heavy chain constant region, an optional linker, and the anti-IL4R scFv, and the third polypeptide chain and the fourth polypeptide chain each comprises, from N- to C-terminus, the anti-TSLP light chain variable region and the light chain constant region, wherein the anti-IL4R scFv in the first and second polypeptide chains each comprises from N- to C-terminus an anti-IL4R heavy chain variable region, an optional linker, and an anti-IL4R light chain variable region, or an anti-IL4R light chain variable region, an optional linker, and an anti-IL4R heavy chain variable region.

In certain embodiments, the first polypeptide chain and the second polypeptide chain each comprises, from N- to C-terminus, the anti-TSLP scFv, an optional linker, the anti-IL4R heavy chain variable region, and the heavy chain constant region, and the third polypeptide chain and the fourth polypeptide chain each comprises, from N- to C-terminus, the anti-IL4R light chain variable region and the light chain constant region, wherein the anti-TSLP scFv in the first and second polypeptide chains each comprises from N- to C-terminus an anti-TSLP heavy chain variable region, an optional linker, and an anti-TSLP light chain variable region, or an anti-TSLP light chain variable region, an optional linker, and an anti-TSLP heavy chain variable region.

In certain embodiments, the first polypeptide chain and the second polypeptide chain each comprises, from N- to C-terminus, the anti-IL4R heavy chain variable region, and the heavy chain constant region, and the third polypeptide chain and the fourth polypeptide chain each comprising, from N- to C-terminus, the anti-TSLP scFv, an optional linker, the anti-IL4R light chain variable region and the light chain constant region, wherein the anti-TSLP scFv in the third and fourth polypeptide chains each comprises from N- to C-terminus an anti-TSLP heavy chain variable region, an optional linker and an anti-TSLP light chain variable region, or an anti-TSLP light chain variable region, an optional linker and an anti-TSLP heavy chain variable region.

In certain embodiments, the first polypeptide chain and the second polypeptide chain each comprises, from N- to C-terminus, the anti-IL4R heavy chain variable region, the heavy chain constant region, an optional linker, and the anti-TSLP scFv, and the third polypeptide chain and the fourth polypeptide chain each comprises, from N- to C-terminus, the anti-IL4R light chain variable region and the light chain constant region, wherein the anti-TSLP scFv in the first and second polypeptide chains each comprises from N- to C-terminus an anti-TSLP heavy chain variable region, an optional linker and an anti-TSLP light chain variable region, or an anti-TSLP light chain variable region, an optional linker and an anti-TSLP heavy chain variable region.

The anti-TSLP binding domain may comprise a heavy chain variable region and a light chain variable region. The heavy chain variable region may comprise a VH CDR1, a VH CDR2 and a VH CDR3 that may comprise the amino acid sequences of SEQ ID NOs: 1, 2 and 3, respectively. The heavy chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 7. The light chain variable region may comprise a VL CDR1, a VL CDR2 and a VL CDR3 that may comprise the amino acid sequences of SEQ ID NOs: 4, 5 and 6, respectively. The light chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 8.

The anti-IL4R binding domain may comprise a heavy chain variable region and a light chain variable region. The heavy chain variable region may comprise a VH CDR1, a VH CDR2 and a VH CDR3 that may comprise the amino acid sequences of SEQ ID NOs: 11, 12 and 13, respectively. The heavy chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 17. The light chain variable region may comprise a VL CDR1, a VL CDR2 and a VL CDR3 that may comprise the amino acid sequences of SEQ ID NOs: 14, 15 and 16, respectively. The light chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 18.

The anti-TSLP binding domain and the anti-IL4R binding domain, may each optionally comprise a heavy chain constant region and a light chain constant region.

The heavy chain constant region may be an IgG1, IgG2, or IgG4 heavy chain constant region, such as human IgG1, IgG2 or IgG4 heavy chain constant region, or a functional fragment thereof, such as an Fc fragment. The heavy chain constant region may be naturally occurring or engineered to have certain desired characteristics. In certain embodiments, the heavy chain constant region is with reduced or eliminated binding affinity to Fc receptors and/or complement system proteins. In certain embodiments, the heavy chain constant region may comprise the amino acid sequence of SEQ ID NOs: 9 or 29. The light chain constant region may be a kappa or lambda light chain constant region. In certain embodiments, the light chain constant region may be human kappa light chain constant region having e.g., the amino acid sequence of SEQ ID NO: 10.

The optional linker may be a peptide linker made up of 5 to 30, 10 to 30, 10 to 20, or 15 amino acids. The linker may be a GS linker, such as -(Gly-Gly-Gly-Gly-Ser)₂- (SEQ ID NO: 19), or -(Gly-Gly-Gly-Gly-Ser)₄- (SEQ ID NO: 20). In certain embodiments, the linker in the scFv is -(Gly-Gly-Gly-Gly-Ser)₄- (SEQ ID NO: 20). In certain embodiments, the linker between the scFv and the IgG heavy/light heavy chain is -(Gly-Gly-Gly-Gly-Ser)₂- (SEQ ID NO: 19).

In certain embodiments, the recombinant bispecific antibody of the disclosure may comprise the first, second, third and fourth polypeptide chains comprising the amino acid sequences having at least 80%, 81%, 82%, 83%, 84%, 85%, 86%, 87%, 88%, 89%, 90%, 91%, 92%, 93%, 94%, 95%, 96%, 97%, 98%, 99% or 100% identity to i) SEQ ID NOs: 21, 21, 39 and 39, respectively; ii) SEQ ID NOs: 22, 22, 39 and 39, respectively; iii) SEQ ID NOs: 23, 23, 40 and 40, respectively; iv) SEQ ID NOs: 24, 24, 40 and 40, respectively; v) SEQ ID NOs: 25, 25, 40 and 40, respectively; vi) SEQ ID NOs: 26, 26, 40 and 40, respectively; vii) SEQ ID NOs: 27, 27, 39 and 39, respectively; or viii) SEQ ID NOs: 28, 28, 39 and 39, respectively.

The present application also provides a nucleic acid molecule encoding the recombinant bispecific antibody of the disclosure, as well as an expression vector that may comprise such a nucleic acid, and a host cell that may comprise such an expression vector or such a nucleic acid molecule, or alternatively have the nucleic acid of the disclosure integrated into its genome. A method for preparing the recombinant bispecific antibody of the disclosure using the host cell is also provided, that may comprise steps of (i) expressing the recombinant bispecific antibody in the host cell and (ii) isolating the recombinant bispecific antibody from the host cell or its cell culture.

The present application also provides a pharmaceutical composition that may comprise the recombinant bispecific antibody, the nucleic acid molecule, the expression vector, or the host cell of the disclosure, and a pharmaceutically acceptable carrier. The pharmaceutical composition may further comprise an additional agent, such as an anti-inflammatory agent.

The present application also provides a kit that may comprise the recombinant bispecific antibody, the nucleic acid molecule, the expression vector, or the host cell of the disclosure.

In a second aspect, the present application provides a method for treating a disease associated with TSLP and/or IL4/IL13 induced or mediated signaling in a subject in need thereof, which may comprise administering to the subject a therapeutically effective amount of the pharmaceutical composition of the disclosure.

The disease may be an inflammatory disease, such as an allergic disease, or an auto-immune disease. The disease may include, but not limited to, atopic dermatitis, asthma, chronic rhinosinusitis, nasal polyposis, and eosinophilic esophagitis.

In certain embodiments, the subject is human.

The disclosure also provides a method for reducing or eliminating an excessive immune response associated with TSLP and/or IL4/IL13 induced or mediated signaling in a subject in need thereof, which may comprise administering to the subject a therapeutically effective amount of the pharmaceutical composition of the disclosure.

The disclosure also provides a method for reducing or eliminating an inflammation associated with TSLP and/or IL4/IL13 induced or mediated signaling in a subject in need thereof, which may comprise administering to the subject a therapeutically effective amount of the pharmaceutical composition of the disclosure.

In certain embodiments, the subject is human.

Also provided is the use of the pharmaceutical composition of the disclosure in treating a disease associated with TSLP and/or IL4/IL13 induced signaling, in reducing or eliminating an excessive immune response associated with TSLP and/or IL4/IL13 induced or mediated signaling, and/or in reducing or eliminating an inflammation associated with TSLP and/or IL4/IL13 induced or mediated signaling; and in preparation of a medicament for treating a disease associated with TSLP and/or IL4/IL13 induced signaling, reducing or eliminating an excessive immune response associated with TSLP and/or IL4/IL13 induced or mediated signaling, and/or reducing or eliminating an inflammation associated with TSLP and/or IL4/IL13 induced or mediated signaling.

Other features and advantages of the instant disclosure will be apparent from the following detailed description and examples, which should not be construed as limiting. The contents of all references, Genbank entries, patents and published patent applications cited throughout this application are expressly incorporated herein by reference.

### BRIEF DESCRIPTION OF THE DRAWINGS

The following detailed description, given by way of example, but not intended to limit the invention solely to the specific embodiments as described, may best be understood in conjunction with the accompanying drawings.

FIG. 1 shows the schematic diagrams of the recombinant bispecific antibodies of the disclosure.

FIGS. 2A-2B show the binding capability of the recombinant bispecific antibodies BSI-502-001 to BSI-502-004 (A) and BSI-502-005 to BSI-502-008 (B) to human TSLP in a capture ELISA.

FIGS. 3A-3B show the binding capability of the recombinant bispecific antibodies BSI-502-001 to BSI-502-004 (A) and BSI-502-005 to BSI-502-008 (B) to human IL4R in a capture ELISA.

FIGS. 4A-4B show the binding capability of the recombinant bispecific antibodies BSI-502-001 to BSI-502-004 (A) and BSI-502-005 to BSI-502-008 (B) to human IL4Rα-expressing cells in a cell-based binding FACS assay.

FIGS. 5A-5B show the binding capability of the recombinant bispecific antibodies BSI-502-001 to BSI-502-004 (A) and BSI-502-005 to BSI-502-008 (B) to human TSLP and human IL4R concurrently in a dual-binding ELISA.

FIGS. 6A-6B show the blocking ability of the recombinant bispecific antibodies BSI-502-001 to BSI-502-004 (A) and BSI-502-005 to BSI-502-008 (B) on TSLP-TSLPR/IL7R binding in a competitive ELISA.

FIGS. 7A-7B show the ability of the recombinant bispecific antibodies BSI-502-001 to BSI-502-004 (A) and BSI-502-005 to BSI-502-008 (B) to block TSLP-cell surface TSLPR/IL7R binding in a cell-based blocking FACS assay.

FIGS. 8A-8B show the blocking ability of the recombinant bispecific antibodies BSI-502-001 to BSI-502-004 (A) and BSI-502-005 to BSI-502-008 (B) on IL4-IL4R binding in a competitive ELISA.

FIGS. 9A-9B show the ability of the recombinant bispecific antibodies BSI-502-001 to BSI-502-004 (A) and BSI-502-005 to BSI-502-008 (B) to block IL4-cell surface IL4Rα binding in a cell-based blocking FACS assay.

FIG. 10 shows the ability of the recombinant bispecific antibodies BSI-502-001 to BSI-502-004 to inhibit TSLP-induced STAT5 signaling in a cell-based reporter assay.

FIG. 11 shows the activity of the recombinant bispecific antibodies BSI-502-001 to BSI-502-004 to inhibit IL4-induced STAT6 phosphorylation in a cell-based assay.

FIG. 12 shows the activity of the recombinant bispecific antibodies BSI-502-001 to BSI-502-004 to inhibit IL13-induced STAT6 phosphorylation in a cell-based assay.

FIG. 13 shows the CCL17 production by human PBMCs induced by TSLP and/or IL4.

FIG. 14 shows the ex vivo inhibitory effect of the recombinant bispecific antibodies BSI-502-001 to BSI-502-004 on CCL-17 production by PBMCs induced by the TSLP-IL4 combination.

### DETAILED DESCRIPTION OF THE INVENTION

The term “TSLP” refers to thymic stromal lymphopoietin. The term “TSLP” comprises variants, isoforms, homologs, orthologs and paralogs. The term “human TSLP” refers to a TSLP protein having an amino acid sequence from a human, such as the amino acid sequence of human TSLP having a Genbank accession number of NP_149024.1 or SEQ ID NO: 32.

The term “IL4Rα” refers to interleukin 4 receptor subunit alpha. The term “IL4Rα” comprises variants, isoforms, homologs, orthologs and paralogs. The term “human IL4Rα” refers to an IL4Rα protein having an amino acid sequence from a human, such as the amino acid sequence of human IL4Rα having a Genbank accession number of NP_001244335.1 or SEQ ID NO: 33.

The term “antibody” as referred to herein includes whole antibodies and any antigen binding fragment (i.e., “antigen-binding portion”) or single chains thereof. Whole antibodies are glycoproteins comprising two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, C_{H1}, C_{H2} and C_{H3}. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and V L is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. The functional fragment of the heavy chain constant region is a part of the constant region that retains desired characteristics, e.g., the ability to bind Fe receptors and/or complement system proteins, and/or the ability to prolong the serum half-life of the antibody or antigen-binding portion thereof.

The term “antigen-binding portion” of an antibody (or simply “antibody portion”), as used herein, refers to one or more fragments of an antibody that retain the ability to specifically bind to an antigen (e.g., an IL4Rα or a TSLP protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules. Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

As used herein, an antibody that “specifically binds to human TSLP” is intended to refer to an antibody that binds to human TSLP protein (and possibly a TSLP protein from one or more non-human species) but does not substantially bind to non-TSLP proteins. Preferably, the antibody binds to human TSLP protein with “high affinity”, namely with a K_{D} of 5.0×10⁻⁸ M or less, more preferably 1.0×10⁻⁸ M or less, and more preferably 1.0×10⁻¹⁰ M or less.

As used herein, an antibody that “specifically binds to human IL4Rα” is intended to refer to an antibody that binds to human IL4Rα protein (and possibly an IL4Rα protein from one or more non-human species) but does not substantially bind to non-IL4Rα proteins. Preferably, the antibody binds to human IL4Rα protein with “high affinity”, namely with a K_{D} of 5.0×10⁻⁸ M or less, more preferably 1.0×10⁻⁸ M or less, and more preferably 5.0×10⁻⁹ M or less.

The term “K_{assoc}” or “Kₐ”, as used herein, is intended to refer to the association rate of a particular antibody-antigen interaction, whereas the term “K_{dis}” or “K_{d}”, as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The term “EC₅₀”, also known as half maximal effective concentration, refers to the concentration of an antibody which induces a response halfway between the baseline and maximum after a specified exposure time.

The term “IC₅₀”, also known as half maximal inhibitory concentration, refers to the concentration of an antibody which inhibits a specific biological or biochemical function by 50% relative to the absence of the antibody.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of the antibody, e.g., the recombinant bispecific antibody, of the present disclosure sufficient to prevent or ameliorate the symptoms associated with a disease or condition (such as an inflammatory disease) and/or lessen the severity of the disease or condition. A therapeutically effective amount is understood to be in context to the condition being treated, where the actual effective amount is readily discerned by those of skill in the art.

The term “identity” as used in the present invention refers to sequence similarity between two polynucleotide sequences or between two amino acid sequences. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof.

The “antagonistic” anti-TSLP antibody refers to an antibody that specifically binds TSLP, blocks TSLP-TSLPR binding/interaction, and suppresses TSLP-induced or mediated signaling. The “antagonistic” anti-IL4R antibody refers to an antibody that specifically binds IL4R, prevents IL4R formation, or blocks IL4R binding with IL4 or IL13, resulting in suppression of IL4 or IL13-induced or mediated signaling.

The recombinant bispecific antibody of the disclosure has comparable binding affinity/activity to TSLP and/or IL4R, comparable blocking activity on IL4-IL4R binding/interaction and/or TSLP-TSLPR/IL7Rα binding/interaction, and/or comparable, if not higher, blocking activity on TSLP and/or IL4 induced or mediated signaling, as compared to the prior art monospecific anti-IL4R or anti-TSLP antibodies such as Dupilumab and Tezepelumab, and even the combination of Dupilumab and Tezepelumab.

The recombinant bispecific antibody of the disclosure may comprise i) an anti-TSLP IgG antibody, comprising a heavy chain and a light chain, and ii) an anti-IL4R scFv linked to N- or C-terminus of the heavy chain or the light chain of the anti-TSLP IgG antibody.

The anti-IL4R scFv may be linked to N-terminus of the heavy chain or the light chain of the anti-TSLP IgG antibody. The anti-IL4R scFv may be linked to N-terminus of each heavy chain or each light chain. The anti-IL4R scFv may be linked to N-terminus of each heavy chain. The anti-IL4R scFv may be linked to N-terminus of each light chain.

Alternatively, the recombinant bispecific antibody of the disclosure may comprise i) an anti-IL4R IgG antibody, comprising a heavy chain and a light chain, and ii) an anti-TSLP scFv linked to the N- or C-terminus of the heavy chain or the light chain of the anti-IL4R IgG antibody.

The anti-TSLP scFv may be linked to N-terminus of the heavy chain or the light chain of the anti-IL4R IgG antibody. The anti-TSLP scFv may be linked to N-terminus of each heavy chain or each light chain. The anti-TSLP scFv may be linked to N-terminus of each heavy chain. The anti-TSLP scFv may be linked to N-terminus of each light chain.

The TSLP binding domain may be an anti-TSLP antibody or an antigen binding portion thereof as described in WO2021/043221, and the IL4R binding domain may be an anti-IL4R antibody or an antigen binding portion thereof described in WO2021/170020. The heavy chain variable region CDRs and the light chain variable region CDRs in these TSLP and IL4R binding domains have been defined by the Kabat numbering system. However, as is well known in the art, CDR regions can also be determined by other systems such as Chothia, IMGT, AbM, or Contact numbering system/method, based on heavy chain/light chain variable region sequences.

The scFv may be linked to the IgG antibody via a linker. The heavy chain variable region in the scFv may be linked to the light chain variable region via a linker. Linkers serve primarily as a spacer between the TSLP binding domain and the IL4R binding domain, or between the heavy and light chain variable regions in the scFv. The linker may be made up of amino acids linked together by peptide bonds, preferably from 5 to 30 amino acids, from 10 to 30 amino acids, from 10 to 20 amino acids, or 15 amino acids, linked by peptide bonds, wherein the amino acids are selected from the 20 naturally occurring amino acids. One or more of these amino acids may be glycosylated, as is understood by those of skill in the art. In one embodiment, the 5 to 30 amino acids may be selected from glycine, alanine, proline, asparagine, glutamine, serine and lysine. In one embodiment, a linker is made up of a majority of amino acids that are sterically unhindered, such as glycine and alanine. Exemplary linkers are polyglycines (particularly (Glys, poly(Gly-Ala), and polyalanine, such as -GGGGSGGGGS- (SEQ ID NO: 19), and -GGGGSGGGGSGGGGSGGGGS- (SEQ ID NO: 20). Linkers may also be non-peptide linkers. For example, alkyl linkers such as —NH—, —(CH₂)ₛ—C(O)—, wherein s=2-20 can be used. These alkyl linkers may further be substituted by any non-sterically hindering group such as lower alkyl (e.g., C₁₋₄) lower acyl, halogen (e.g., CI, Br), CN, NH2, phenyl, etc.

The recombinant bispecific antibody of the disclosure may be engineered by modifying one or more residues within one or both variable regions (i.e., VH and/or VL), for example within one or more CDR regions and/or within one or more framework regions, to e.g., change target binding affinity. Additionally or alternatively, the bispecific antibody can be engineered by modifying the residues within the constant region(s), for example to alter the effector function(s) of the bispecific antibody.

For example, the bispecific antibodies of the disclosure can be engineered to include modifications within the Fc region, typically to alter one or more functional properties of the bispecific antibody, such as serum half-life, complement fixation, Fe receptor binding, and/or antigen-dependent cellular cytotoxicity. Furthermore, a bispecific antibody of the disclosure can be chemically modified (e.g., one or more chemical moieties can be attached to the antibody) or be modified to alter its glycosylation, again to alter one or more functional properties.

In one embodiment, the hinge region of C_{H1} is modified in such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to increase or decrease the stability of the antibody.

In another embodiment, the Fc hinge region in the bispecific antibody of the disclosure is mutated to decrease the biological half-life of the antibody. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of the bispecific antibody is modified. For example, a glycosylated bispecific antibody can be made (i.e., the bispecific antibody lacks glycosylation). Glycosylation can be altered to, for example, increase the affinity of the anti-CD40 antibody or antigen binding portion thereof for the antigen.

Another modification of the bispecific antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antigen binding portion thereof.

In another aspect, the disclosure provides a nucleic acid molecule that encodes the bispecific antibody of the disclosure or portions thereof. For example, the nucleic acid molecule of the disclosure may encode an anti-IL4R heavy chain variable region-heavy chain constant region-anti-TSLP scFv chain, an anti-TSLP scFv-anti-IL4R heavy chain variable region-heavy chain constant region chain, an TSLP heavy chain variable region-heavy chain constant region-anti-IL4R scFv chain, or an anti-IL4R scFv-anti-TSLP heavy chain variable region-heavy chain constant region chain.

The nucleic acids can be present in whole cells, in a cell lysate, or in a partially purified or substantially pure form. A nucleic acid is “isolated” or “rendered substantially pure” when purified from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a DNA molecule.

Nucleic acids of the disclosure can be obtained using standard molecular biology techniques. For example, the nucleic acid molecules of the disclosure may be chemically synthesized.

The present disclosure also provides an expression vector comprising the nucleic acid molecules of the disclosure. Examples of vectors include but are not limited to plasmids, viral vectors, yeast artificial chromosomes (YACs), bacterial artificial chromosomes (BACs), transformation-competent artificial chromosomes (TACs), mammalian artificial chromosomes (MACs) and human artificial episomal chromosomes (HAECs). The present disclosure further provides host cells comprising the expression vectors of the disclosure or having the nucleic acid molecules integrated into their genomes. The host cells may be transformed or transfected with the expression vectors. Suitable host cells include Escherichia coli, yeasts and other eukaryotes. In one embodiment, DNAs encoding the polypeptide chains forming each bispecific antibody of the disclosure are inserted into one or more expression vectors such that the genes are operatively linked to transcriptional and translational regulatory sequences. In this context, the term “operatively linked” is intended to mean that the coding nucleotides are ligated into a vector such that transcriptional and translational control sequences within the vector serve their intended functions of regulating the transcription and translation of the antibody gene(s).

The term “regulatory sequence” is intended to include promoters, enhancers and other expression control elements (e.g., polyadenylation signals) that control the transcription or translation of the nucleotides. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyomavirus enhancer. Alternatively, non-viral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

In addition to the bispecific antibody coding nucleotides and regulatory sequences, the expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the peptide chains constituting the bispecific antibodies, the expression vector(s) encoding the peptide chains is(are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the bispecific antibodies of the disclosure in either prokaryotic or eukaryotic host cells, expression in eukaryotic cells, and most preferably mammalian host cells, is the most preferred because such eukaryotic cells, and in particular mammalian cells, are more likely than prokaryotic cells to assemble and secrete a properly folded and immunologically active bispecific antibody.

Preferred mammalian host cells for expressing the recombinant antibodies of the disclosure include Chinese Hamster Ovary (CHO cells) (including dhfr-CHO cells, described in Urlaub and Chasm, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors are introduced into mammalian host cells, the bispecific antibodies are produced by culturing the host cells for a period of time sufficient to allow for expression of the bispecific antibody in the host cells or, more preferably, secretion of the bispecific antibody into the culture medium in which the host cells are grown. The bispecific antibodies can be recovered from the culture medium using standard protein purification methods.

In another aspect, the present disclosure provides a pharmaceutical composition which may comprise the recombinant bispecific antibody, the nucleic acid molecule, the expression vector, and/or the host cell of the present disclosure formulated together with a pharmaceutically acceptable carrier. The bispecific antibody, the nucleic acid molecule, the expression vector, and/or the host cell can be dosed separately when the composition contains more than one antibody, nucleic acid molecule, expression vector or host cell. The composition may optionally contain one or more additional pharmaceutically active ingredients, such as another antibody or a drug, such as an anti-tumor drug.

The pharmaceutical composition may comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the composition, the dosage may range from about 0.0001 to 100 mg/kg.

A “therapeutically effective dosage” of the bispecific antibody, nucleic acid molecule, expression vector or host cell of the disclosure preferably results in a decrease in severity of disease symptoms, an increase in frequency and duration of disease symptom-free periods, or a prevention of impairment or disability due to the disease affliction. For example, for the treatment of subjects with an inflammatory disease, a “therapeutically effective dosage” preferably eliminate inflammations by at least about 20%, more preferably by at least about 40%, even more preferably by at least about 60%, and still more preferably by at least about 80% relative to untreated subjects.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

The pharmaceutical composition which may comprise the bispecific antibodies, nucleic acid molecules, expression vectors or host cells of the present disclosure have numerous in vitro and in vivo utilities involving, for example, treatment of diseases with TSLP and/or IL4/IL13 induced or mediated signaling.

The disclosure provides a method for treating a disease associated with TSLP and/or IL4/IL13 induced or mediated signaling in a subject in need thereof, which may comprise administering to the subject a therapeutically effective amount of the pharmaceutical composition of the disclosure.

The disease may be an inflammatory disease, such as an allergic disease, or an auto-immune disease. The disease may include, but not limited to, atopic dermatitis, asthma, ulcerative colitis, psoriasis, nasal polyps, and rhinosinusitis. In certain embodiments, the subject is human.

The disclosure also provides a method for reducing or eliminating an excessive immune response associated with TSLP and/or IL4/IL13 induced or mediated signaling in a subject in need thereof, which may comprise administering to the subject a therapeutically effective amount of the pharmaceutical composition of the disclosure. The disclosure also provides a method for reducing or eliminating inflammations associated with TSLP and/or IL4/IL13 induced or mediated signaling in a subject in need thereof, which may comprise administering to the subject a therapeutically effective amount of the pharmaceutical composition of the disclosure.

Although the present invention and its advantages have been described in detail, it should be understood that various changes, substitutions and alterations can be made herein without departing from the spirit and scope of the invention as defined in the appended claims.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1. Construction and Expression of Exemplary Recombinant Bispecific Antibodies

Bispecific antibodies, including BSI-502-001, BSI-502-002, BSI-502-007, and BSI-502-008, were constructed by linking an anti-TSLP scFv, via a linker, to the N- or C-terminus of the heavy chain of a humanized anti-IL4R antibody huC2C1A1A1-V15. The antibody huC2C1A1A1-V15 was described in WO2021/170020 and contained a human IgG heavy chain constant region. The anti-TSLP scFv contained heavy and light chain variable regions from a humanized anti-TSLP antibody hu1C5F12E9-V8 as described in WO2021/043221.

Bispecific antibodies, BSI-502-003, BSI-502-004, BSI-502-005, and BSI-502-006, were constructed by linking an anti-IL4R scFv, with the heavy and light chain variable regions from huC2C1A1A1-V15, via a linker, to the N- or C-terminus of the heavy chain of hu1C5F12E9-V8 with human IgG heavy chain constant region.

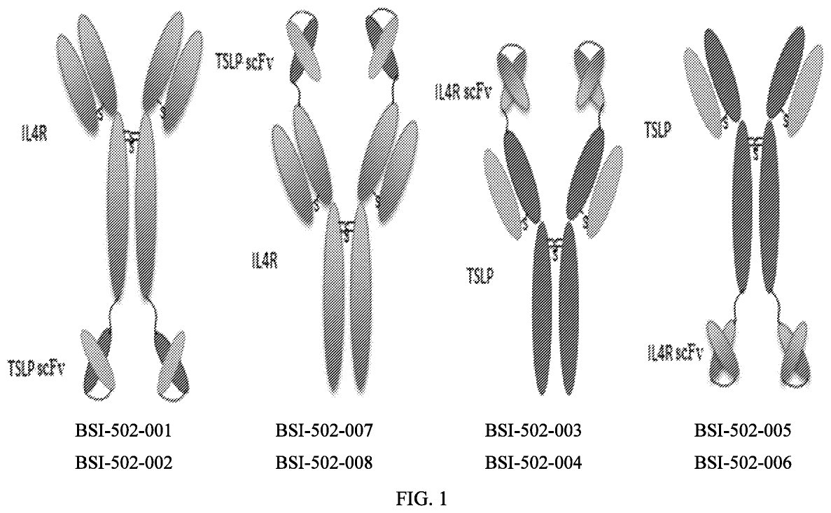

See Table 1 and FIG. 1 for the detailed structures and sequences of these bispecific antibodies.

Monospecific antibodies, i.e., the anti-TSLP hu1C5F12E9-V8 and the anti-IL4R huC2C1A1A1-V15, were prepared as IgG1 or IgG4 typed full-length antibodies and used as controls in the assays below. In specific, hu1C5F12E9-V8 contained a heavy chain variable region, a light chain variable region, a heavy chain constant region, and a light chain constant region having the amino acid sequences of SEQ ID NOs: 7, 8, 9 (IgG1) or 29 (IgG4), and 10, respectively. The huC2C1A1A1-V15 contained a heavy chain variable region, a light chain variable region, a heavy chain constant region, and a light chain constant region having the amino acid sequences of SEQ ID NOs: 17, 18, 9 (IgG1) or 29 (IgG4), and 10, respectively.

In both the bispecific and monospecific antibodies, the heavy chain constant region was prepared with L234A, L235Q, and K322Q (abbreviated as AQQ, to reduce Fc effector functions) and M252Y, S254T, and T256E substitutions (abbreviated as YTE, to increase the half-life).

TABLE 1
Constituent polypeptide chains of Exemplary Bispecific Antibodies

| Antibody ID | Constituent polypeptide chains | |
|---|---|---|
| BSI-502-001 | anti-IL4R heavy chain-linker- | anti-IL4R light chain (SEQ ID NO: 39) |
| | anti-TSLP V_{H}-V_{L} (SEQ ID NO: 21) | |
| BSI-502-002 | anti-IL4R heavy chain-linker- | anti-IL4R light chain (SEQ ID NO: 39) |
| | anti-TSLP V_{L}-V_{H} (SEQ ID NO: 22) | |
| BSI-502-003 | anti-IL4R V_{H}-V_{L}-linker-anti- | anti-TSLP light chain (SEQ ID NO: 40) |
| | TSLP heavy chain (SEQ ID NO: 23) | |
| BSI-502-004 | anti-IL4R V_{L}-V_{H}-linker-anti- | anti-TSLP light chain (SEQ ID NO: 40) |
| | TSLP heavy chain (SEQ ID NO: 24) | |
| BSI-502-005 | anti-TSLP heavy chain-linker- | anti-TSLP light chain (SEQ ID NO: 40) |
| | anti-IL4R V_{H}-V_{L} (SEQ ID NO: 25) | |
| BSI-502-006 | anti-TSLP heavy chain-linker-anti- | anti-TSLP light chain (SEQ ID NO: 40) |
| | IL4R V_{L}-V_{H} (SEQ ID NO: 26) | |
| BSI-502-007 | anti-TSLP V_{H}-V_{L}-linker-anti- | anti-IL4R light chain (SEQ ID NO: 39) |
| | IL4R heavy chain (SEQ ID NO: 27) | |
| BSI-502-008 | anti-TSLP V_{L}-V_{H}-linker-anti- | anti-IL4R light chain (SEQ ID NO: 39) |
| | IL4R heavy chain (SEQ ID NO: 28) | |

Nucleic acids encoding the polypeptide chains constituting the bispecific antibodies were synthesized, and then inserted into pTT5 plasmids, respectively. Afterwards, the resultant plasmids were transfected into CHO cells, and the exemplary bispecific antibodies of the disclosure were expressed and secreted by the CHO cells, which were purified later using Protein A sepharose columns.

### Example 2. Binding Affinity Determination of Exemplary Bispecific Antibodies Using BIACORE Surface Plasmon Resonance Technology

The purified bispecific antibodies were characterized for their binding affinity and binding kinetics by Biacore T200 system (GE healthcare, Pittsburgh, PA, USA), with huC2C1A1A-V15(IgG1), Dupilumab (an anti-IL4Rα antibody), hu1C5F12E9-V8(IgG1), and Tezepelumab (an anti-TSLP antibody, prepared in-house with heavy and light chains of SEQ ID NOs: 30 and 31) used as positive controls.

The goat anti-human IgG (GE healthcare, Cat #BR100839, Human Antibody Capture Kit) was covalently linked to a CM5 chip (carboxy methyl dextran coated chip) via primary amines, using a standard amine coupling kit provided by Biacore (GE healthcare, Pittsburgh, PA, USA). Un-reacted moieties on the biosensor surface were blocked with ethanolamine. Then, purified bispecific antibodies of the disclosure at the concentration of 66.67 nM and the controls at 10 μg/mL, were flowed onto the chip at a flow rate of 10 μL/min. Then, serially diluted recombinant human IL4Rα-his (in house made, IL4Rα of SEQ ID NO: 33 with His tag at C-terminus) in HBS EP buffer (provided by Biacore) was flowed onto the chip at a flow rate of 30 μL/min. The association and dissociation curves were fit to a 1:1 Langmuir binding model using BIAcore evaluation software. The K_{D}, Kₐ and K_{d} values were determined and shown in Table 2-1 below.

TABLE 2-1
Binding Affinity of Bispecific Antibodies to Human IL4R

| | Kinetics on Biacore | | |
|---|---|---|---|
| | Human IL4Rα-his | | |
| | Kₐ | K_{d} | K_{D} |
| Sample | (M⁻¹s⁻¹) | (s⁻¹) | (M) |
| BSI-502-001 | 1.61E+05 | 4.97E−04 | 3.09E−09 |
| BSI-502-002 | 3.23E+05 | 4.87E−04 | 1.51E−09 |
| BSI-502-003 | 2.20E+05 | 5.34E−04 | 2.43E−09 |
| BSI-502-004 | 1.86E+05 | 4.53E−04 | 2.43E−09 |
| BSI-502-005 | 5.49E+04 | 1.61E−04 | 2.93E−09 |
| BSI-502-006 | 5.00E+04 | 2.44E−04 | 4.88E−09 |
| BSI-502-007 | 1.80E+05 | 3.99E−04 | 2.22E−09 |
| BSI-502-008 | 1.38E+05 | 4.36E−04 | 3.15E−09 |
| Dupilumab | 2.85E+05 | 5.58E−05 | 1.96E−10 |
| huC2C1A1A1-V15(IgG1) | 1.68E+05 | 3.48E−04 | 2.07E−09 |

TABLE 2-2
Binding Affinity of Bispecific Antibodies to Human TSLP

Kinetics on Biacore
Human TSLP-his

| | Kₐ | K_{d} | K_{D} |
|---|---|---|---|
| Sample | (M⁻¹s⁻¹) | (s⁻¹) | (M) |
| BSI-502-001 | 4.07E+05 | 1.99E−08 | 4.88E−14 |
| BSI-502-002 | 2.12E+05 | 6.51E−09 | 3.07E−14 |
| BSI-502-003 | 5.74E+05 | 1.82E−07 | 3.17E−13 |
| BSI-502-004 | 4.99E+05 | 1.71E−06 | 3.42E−12 |
| BSI-502-005 | 6.92E+05 | 2.17E−08 | 3.14E−14 |
| BSI-502-006 | 7.10E+05 | 1.43E−09 | 2.02E−15 |
| BSI-502-007 | 4.60E+05 | 3.87E−06 | 8.40E−12 |
| BSI-502-008 | 5.85E+05 | 4.43E−06 | 7.58E−12 |
| Tezepelumab | 1.43E+06 | 1.14E−05 | 7.95E−12 |
| hu1C5F12E9-V8(IgG1) | 6.36E+05 | 2.42E−08 | 3.81E−14 |

Biosion in house synthesized recombinant human TSLP-his (TSLP of SEQ ID NO: 32 with His tag at C-terminus) at the concentration of 10 μg/mL was covalently linked to a CM5 chip (carboxy methyl dextran coated chip from GE healthcare #BR100530) via primary amines, using a standard amine coupling kit provided by Biacore (GE healthcare, Pittsburgh, PA, USA). Un-reacted moieties on the biosensor surface were blocked with ethanolamine. Then, serially diluted purified bispecific antibodies and the controls (2-fold serial dilution in HBS-EP⁺ buffer starting at 100 nM) were respectively flowed onto the chip at a flow rate of 30 μL/min. The antigen-antibody association kinetics was followed for 4 minutes and the dissociation kinetics was followed for 13 minutes. The association and dissociation curves were fit to a 1:1 Langmuir binding model using BIAcore evaluation software, and the K_{D}, Kₐ and K_{d} values were determined and shown in Table 2-2.

All the bispecific antibodies of the disclosure specifically bound to human TSLP and human IL4R proteins with high binding affinity.

### Example 3. Binding Activity of Exemplary Bispecific Antibodies

The binding activity of the exemplary bispecific antibodies of the disclosure to human TSLP or human IL4Rα was determined by Capture ELISA and Flow Cytometry (FACS).

### 3.1 Capture ELISA for TSLP Binding

Briefly, 96-well micro plates were coated with 100 μL 2 μg/mL AffiniPure F(ab′)₂ Fragment Goat Anti-Human IgG, Fey fragment specific (Jackson Immuno Research, Cat #109-006-008) in PBS and incubated overnight at 4° C. Plates were washed 4 times with wash buffer (PBS+0.05% Tween-20, PBST) and then blocked with 200 μL/well blocking buffer (5% w/v non-fatty milk in PBST) for 2 hours at 37° C. Plates were washed again and incubated with 100 μL serially diluted bispecific antibodies of the present disclosure, the positive controls or hIgG (Hualan Biological Engineering Inc.), 5-fold serial dilution in 2.5% non-fatty milk in PBST starting at 60 nM, for 40 minutes at 37° C., and then washed 4 times again. Plates containing captured antibodies were incubated with 100 μL biotin-labeled human TSLP-his proteins (TSLP of SEQ ID NO: 32 with His tag at the C-terminus, prepared in-house, 35 ng/mL in 2.5% non-fatty milk in PBST) for 40 minutes at 37° C., washed 4 times, and incubated with streptavidin conjugated HRP (1:10000 dilution in PBST, Jackson Immuno Research, Cat #016-030-084, 100 μL/well) for 40 minutes at 37° C. After the final wash, the plates were incubated with 100 μL/well TMB (Innoreagents). The reaction was stopped 15 minutes later at room temperature with 50 μL/well 1M H₂SO₄, and the absorbance of each well was read on a microplate reader using dual wavelength mode with 450 nm for TMB and 630 nm as the reference wavelength. The OD (450-630) values were plotted against antibody concentration. Data was analyzed using Graphpad Prism and EC₅₀ values were reported.

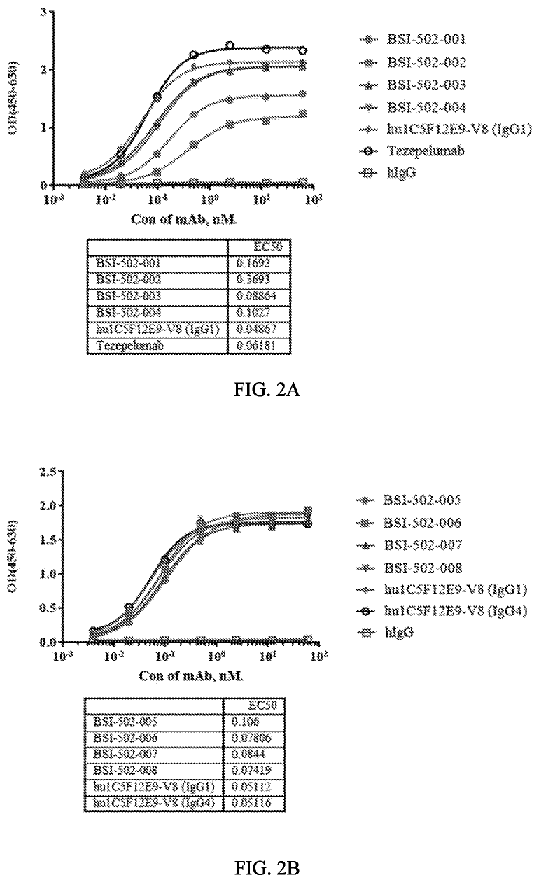

It can be seen from FIGS. 2A and 2B that bispecific antibodies of the disclosure specifically bound to human TSLP, and the Bmax (maximal binding) and EC₅₀ were comparable with those of the positive controls, except for BSI-502-002.

### 3.2 Capture ELISA for IL4R Binding

Briefly, 96-well plates were coated with 2 μg/mL AffiniPure F(ab′)₂ Fragment Goat Anti-Human IgG, Fey fragment specific (Jackson Immuno Research, Cat #109-006-008) in PBS, 100 μL/well, overnight at 4° C. Plates were washed once with wash buffer (PBS+0.05% w/v Tween-20, PBST) and then blocked with 200 μL/well blocking buffer (5% w/v non-fatty milk in PBST) for 2 hours at 37° C. Plates were washed again and incubated with 100 μL/well serially diluted bispecific antibodies of the disclosure, the positive controls or negative control hIgG (human immunoglobulin (pH 4) for intravenous injection, Hualan Biological Engineering Inc.) (5-fold dilution in 2.5% w/v non-fatty milk in PBST, starting at 20 nM) for 40 minutes at 37° C., and then washed 4 times again. Plates containing captured antibodies were incubated with biotin-labeled human IL4Rα-his protein (IL4Rα of SEQ ID NO: 33 with His tag at C terminus, 6.8 ng/mL in 2.5% w/v non-fatty milk in PBST, 100 μL/well) for 40 minutes at 37° C., washed 4 times, and incubated with streptavidin conjugated HRP (1:10000 dilution in PBST, Jackson Immuno Research, Cat #016-030-084, 100 μL/well) for 40 minutes at 37° C. After a final wash, plates were incubated with 100 μL/well ELISA substrate TMB (Innoreagents, Cat #TMB-S-002). The reaction was stopped in 10 minutes at 25° C. with 50 μL/well 1M H₂SO₄, and the absorbance was read on a microplate reader using dual wavelength mode with 450 nm for TMB and 630 nm as the reference wavelength. Data was analyzed using Graphpad Prism and EC₅₀ values were reported.

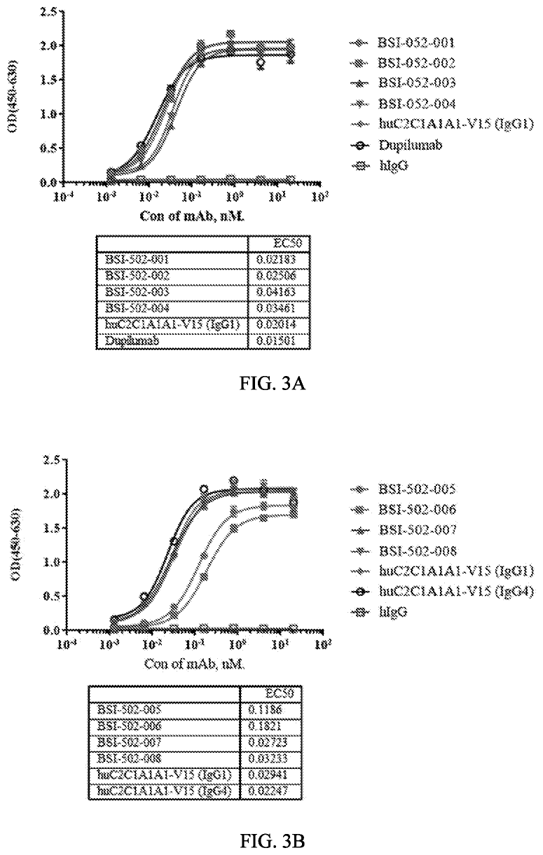

According to FIGS. 3A and 3B, the bispecific antibodies of the disclosure specifically bound to human IL4R. The Bmax and EC₅₀ were close to those of the positive controls, except for BSI-502-005 and BSI-502-006.

### 3.3 Cell-Based Binding FACS

The binding activity of the bispecific antibodies to human IL4Rα expressed on 293F-IL4Rα cell surface was tested by flow cytometry (FACS). Briefly, 293F cells (Thermofisher Inc., Cat #11625019) were transfected with a pCMV-T-P plasmid construct with the nucleotide encoding human IL4Rα (amino acid residues 1-825 of uniprot #P24394-1) inserted between EcoRI and XbaI, and a stable cell pool named 293F-IL4Rα was chosen for subsequent cell-based binding and cell-based ligand blocking FACS assays. The 293F-IL4Rα cells were harvested from cell culture flasks, washed twice and resuspended in phosphate buffered saline (PBS) containing 2% v/v Fetal Bovine Serum (FACS buffer). Then, 2×10⁵ cells per well were incubated in 96 well-plates with 100 μL/well serially diluted bispecific antibodies, positive controls or hIgG (starting at 100 nM with a 5-fold serial dilution) in FACS buffer for 40 minutes on ice. Cells were washed twice with FACS buffer, and added with 100 μL/well R-Phycoerythrin AffiniPure Goat Anti-Human IgG, Fey fragment specific (1:1000 dilution in FACS buffer, Jackson Immunoresearch, Cat #109-115-098). Following an incubation of 40 minutes at 4° C. in dark, cells were washed three times and resuspended in FACS buffer. Fluorescence was measured using a Becton Dickinson FACS Canto II-HTS equipment. Data was analyzed using Graphpad Prism and EC₅₀ values were reported.

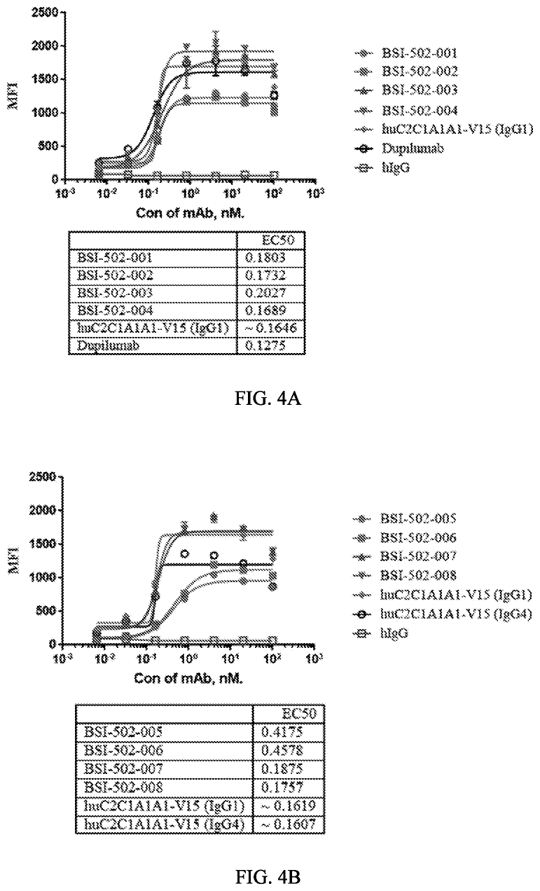

According to FIGS. 4A and 4B, the bispecific antibodies of the disclosure specifically bound to human IL4R expressed on cell surface and EC₅₀ was comparable to those of the positive controls, except for BSI-502-005 and BSI-502-006.

### 3.4 Dual-Binding ELISA

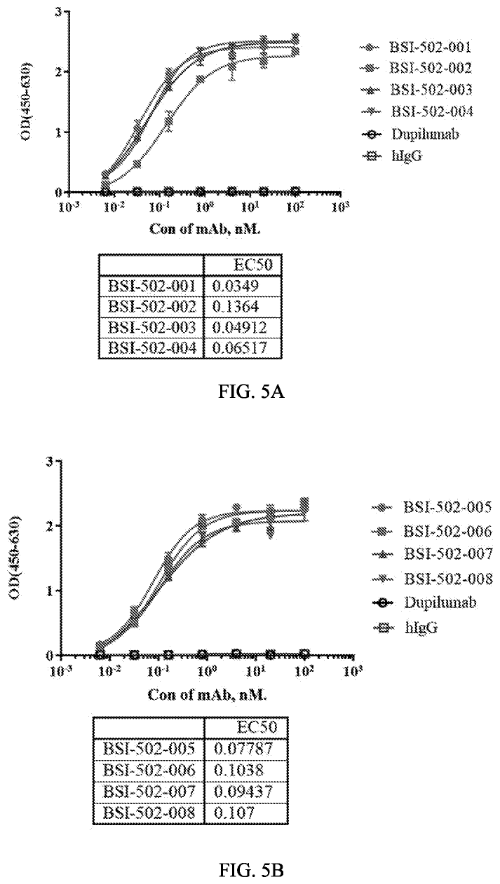

For the dual-binding ELISA, 96-well ELISA plates were firstly coated with 100 μL 1 μg/mL human IL4Rα-his protein (IL4Rα of SEQ ID NO: 33 with His tag at C terminus, prepared in-house) in PBS overnight at 4° C. ELISA plates were washed 4 times with wash buffer (PBS+0.05% Tween-20, PBST) and then blocked with 200 μL/well blocking buffer (5% w/v non-fatty milk in PBST) for 2 hours at 37° C. Plates were washed again and incubated with 100 μL serially diluted bispecific antibodies of the disclosure, positive controls or hIgG (5-fold serial dilution in 2.5% non-fatty milk in PBST starting at 100 nM) for 40 minutes at 37° C. ELISA plates were washed 4 times again and incubated with biotin-labelled human TSLP-his solution (TSLP of SEQ ID NO: 32 with His tag at the C terminus, 1:2000 dilution, final concentration at 0.175 μg/mL in PBST buffer, 100 μL/well) for 40 minutes at 37° C. ELISA plates were washed again and incubated with SA-HRP (100 μl/well) for 40 minutes at 37° C. After the final wash, 100 μL/well TMB (Innoreagents) was added. The reaction was stopped 15 minutes later at room temperature with 50 μL/well 1M H₂SO₄, and the absorbance was read on a microplate reader using dual wavelength mode with 450 nm for TMB and 630 nm as the reference wavelength. The OD (450-630) values were plotted against antibody concentration. Data was analyzed using Graphpad Prism and EC₅₀ values were reported. The results were shown in FIGS. 5A and 5B.

The results showed that the bispecific antibodies of the disclosure bound IL4R and TSLP concurrently, whereas the monospecific antibody Dupilumab exhibited no binding signal.

### Example 4. Blocking Activity of Exemplary Bispecific Antibodies on IL4R-IL4 or TSLPR/IL7Ra-TSLP Binding 4.1 Inhibitory Effect on TSLPR/IL7Ra-TSLP Binding in Ligand Blocking ELISA

The ability of the bispecific antibodies to block TSLP-TSLPR/IL7Ra binding was measured using a competitive ELISA assay. Briefly, 100 μL human TSLPR-Fc proteins (SEQ ID NO: 34-SEQ ID NO: 38 fusion, prepared in-house) at 1 μg/mL in PBS, and 100 μL human IL7Ra-Fc proteins (SEQ ID NO: 35-SEQ ID NO: 38 fusion, prepared in-house) at 1 μg/mL in PBS were coated on 96-well micro plates overnight at 4° C. The next day, plates were washed with wash buffer (PBS+0.05% Tween-20, PBST), and blocked with 5% w/v non-fatty milk in PBST for 2 hours at 37° C. Plates were then washed again using wash buffer.

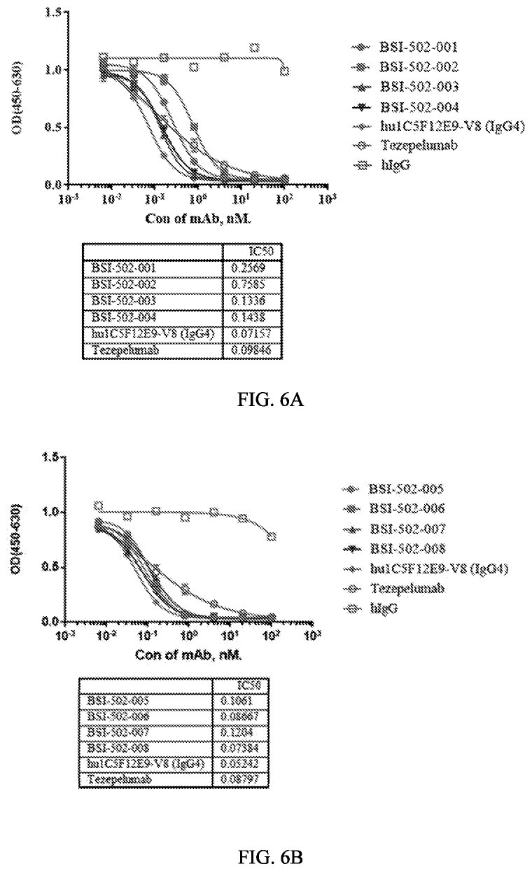

The bispecific antibodies of the disclosure or controls were diluted in biotin-labeled human TSLP-his (TSLP of SEQ ID NO: 32 with C-terminal His tag, prepared in-house, 17 ng/mL in 2.5% non-fatty milk in PBST), starting at 100 nM with a 5-fold serial dilution, and incubated at room temperature for 40 minutes. Then 100 μL of the antibody/TSLP-his mixtures were added to TSLPR/IL7Ra-coated plates. After incubation at 37° C. for 40 minutes, plates were washed 4 times using wash buffer. Then streptavidin conjugated HRP was added and incubated for 40 minutes at 37° C. to detect biotin-labeled human TSLP-his bound to TSLPR/IL7R. Plates were washed again using wash buffer. Finally, TMB was added and the reaction was stopped using 1M H₂SO₄. The absorbance was read on a microplate reader using dual wavelength mode with 450 nm for TMB and 630 nm as the reference wavelength, then the OD (450-630) values were plotted against the antibody concentration. Data was analyzed using Graphpad Prism and IC₅₀ values were reported. The results were shown in FIGS. 6A and 6B.

It can be seen from FIGS. 6A and 6B that all the bispecific antibodies of the disclosure were capable of blocking human TSLP binding to human TSLPR/IL7Ra with comparable activity to the positive controls.

### 4.2 Inhibitory Effect on TSLPR/IL7Ra-TSLP Binding in Cell-Based Ligand Blocking FACS

The activity of bispecific antibodies to block the binding of TSLP-Fc protein to cell surface human TSLPR/human IL7Ra was evaluated in a Flow Cytometry (FACS) assay, using HEK293T-TSLPR/IL7R/STAT5-Luc 5C5 cells expressing cell-surface human TSLPR (SEQ ID NO: 36) and human IL7Ra (SEQ ID NO: 37). The cells were prepared following the instruction of lipofectamine 3000 transfection reagent (Thermo Fisher), by transfecting HEK293T cells (ATCC® CRL-11268) with pCMV-T-P plasmids inserted with TSLPR coding sequence between EcoRI and Xba1 sites and pCMV3-SP plasmids inserted with IL7Ra coding sequence between HindIII and Xba1 sites as well as pGL4.52 [luc2P/STAT5RE/Hygro](Promega).

Briefly, the bispecific antibodies of the disclosure, the positive controls or negative control hIgG (human immunoglobulin (pH4) for intravenous injection, Hualan Biological Engineering Inc.) were diluted with biotin-labeled human TSLP-Fc solution (SEQ ID NO: 32-SEQ ID NO: 38 fusion, prepared in-house, 0.29 μg/mL in FACS buffer), 5-fold serial dilution starting at 100 nM, and incubated at room temperature for 40 minutes. The cells were harvested from cell culture flasks, washed twice and re-suspended in phosphate buffered saline (PBS) containing 2% v/v Fetal Bovine Serum (FACS buffer). Then, 1×10⁵ HEK293T-TSLPR/IL7R/STAT5-Luc 5C5 cells per well were incubated in 96 well-plates with 100 μL/well antibody/TSLP-Fc-biotin mixtures for 40 minutes at 4° C. Cells were washed twice with FACS buffer, and then added and incubated with 100 μL/well R-Phycoerythrin Streptavidin (1:500 dilution in FACS buffer, Jackson Immunoresearch, Cat #016-110-084) for 40 minutes at 4° C. in dark. Cells were washed twice and re-suspended in FACS buffer. Fluorescence was measured using a Becton Dickinson FACS Canto II-HTS equipment. Data was analyzed using Graphpad Prism and IC₅₀ values were reported.

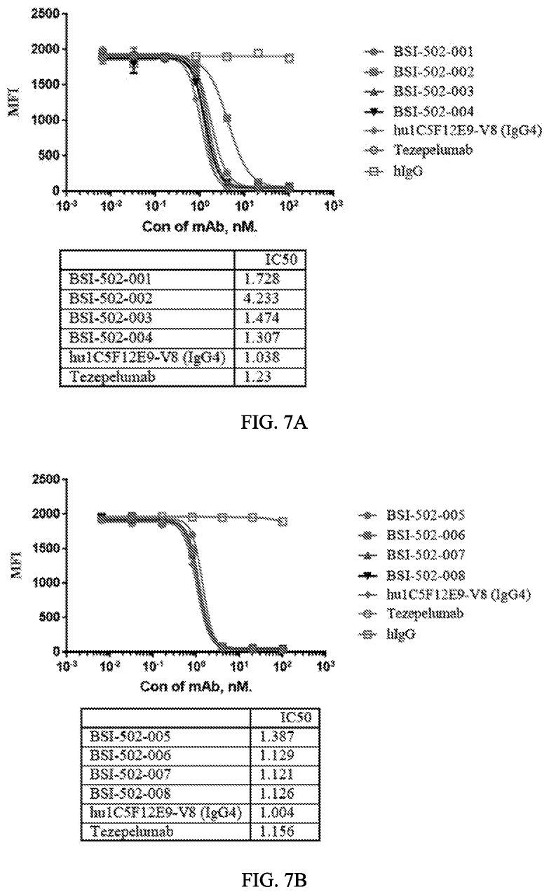

As shown in FIGS. 7A and 7B, the bispecific antibodies of the disclosure completely inhibited TSLP-TSLPR/IL7Ra binding with similar IC₅₀ as compared with the positive controls, with the inhibition effect of BSI-502-002 a bit lower than those of the positive controls.

### 4.3 Inhibitory Effect on IL4-IL4R Binding in Ligand Blocking ELISA

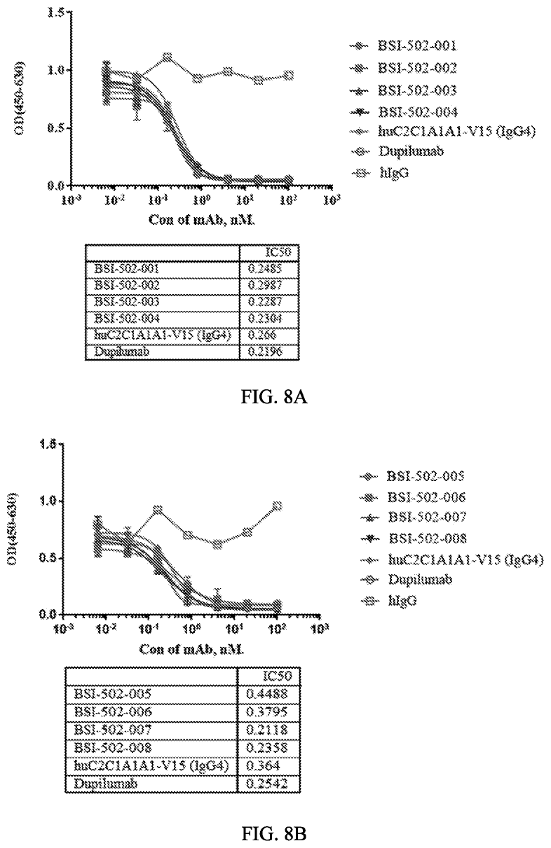

The ability of the bispecific antibodies of the disclosure to block IL4-IL4Rα interaction was measured in a competitive ELISA assay. Briefly, 100 μL human IL4Rα-his proteins (prepared in-house, IL4Rα of SEQ ID NO: 33 with C-terminal His tag) were coated on 96-well micro plates at 2 μg/mL in PBS overnight at 4° C. The next day, plates were washed with wash buffer (PBS+0.05% w/v Tween-20, PBST), and blocked with 5% w/v non-fatty milk in PBST for 2 hours at 37° C. Plates were then washed again using wash buffer. Serially diluted bispecific antibodies or the controls (starting at 100 nM with a 5-fold serial dilution) in 2.5% w/v non-fatty milk in PBST, 100 μL per well, were added to the IL4Rα bound plates, and incubated at 37° C. for 40 minutes. Plates were washed 4 times using wash buffer, and then added and incubated for 40 minutes at 37° C. with 100 μL/well of 0.3 μg/mL biotin-labeled human IL4 protein (Sino biological inc., Cat #11846-HNAE). Plates were washed again using wash buffer. Thereafter, the plates were added with 100 μL/well of streptavidin conjugated HRP (1:10000 dilution in PBST buffer, Jackson Immunoresearch, Cat #016-030-084) and incubated for 40 minutes at 37° C. Plates were washed again using wash buffer. Finally, TMB was added and the reaction was stopped using 1M H₂SO₄, and the absorbance was read at 450 nm. Data was analyzed using Graphpad Prism and IC₅₀ values were reported. The results were shown in FIGS. 8A and 8B.

It can be seen from FIGS. 8A and 8B that the bispecific antibodies of the disclosure were capable of blocking human IL4 binding to human IL4R, with comparable activity to the positive controls.

### 4.4 Inhibitory Effect on IL4R-IL4 Binding in Cell-Based Ligand Blocking FACS

The activity of the bispecific antibodies to block IL4 protein binding to cell surface human IL4Rα was evaluated by Flow Cytometry (FACS), using the 293F-IL4Rα cells mentioned above.

Briefly, the 293F-IL4Rα cells were harvested from cell culture flasks, washed twice and resuspended in PBS containing 2% v/v Fetal Bovine Serum (FACS buffer). Then, 1×10⁵ cells per well were incubated in 96 well-plates in 100 μL of serially diluted bispecific antibodies, the positive controls or negative control (starting at 100 nM with a 5-fold serial dilution) in FACS buffer for 40 minutes on ice. The plates were washed twice with FACS buffer, and added and incubated for 40 minutes at 4° C. in dark with 100 μL/well 0.3 μg/mL biotin-labeled human IL4 protein (Sino biological inc., Cat #11846-HNAE). The plates were washed twice with FACS buffer, and then added and incubated for 40 minutes at 4° C. in dark with 100 μL/well R-Phycoerythrin Streptavidin (1:500 dilution in FACS buffer, Jackson Immunoresearch, Cat #016-110-084). Cells were washed twice and resuspended in FACS buffer. Fluorescence was measured using a Becton Dickinson FACS Canto II-HTS equipment. Data was analyzed using Graphpad Prism and IC₅₀ values were reported.

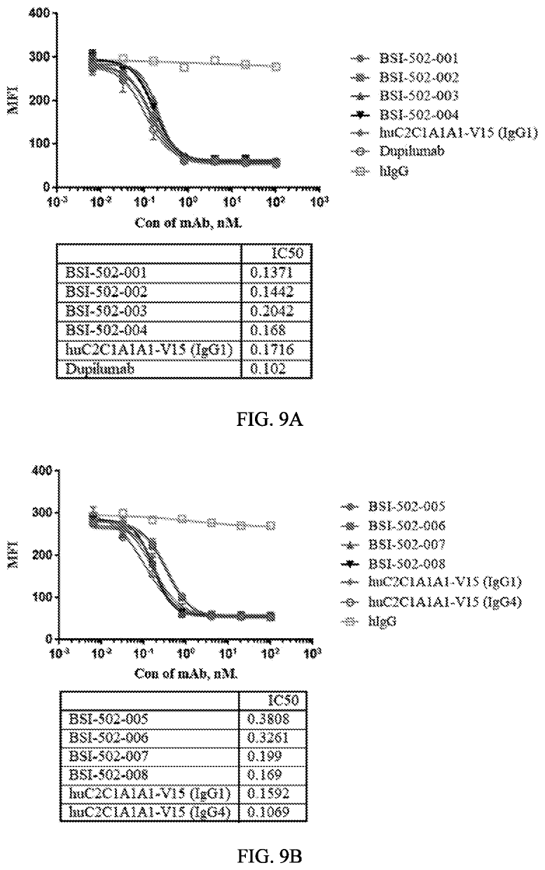

As shown in FIGS. 9A and 9B that all the bispecific antibodies of the disclosure were able to block IL4 binding to cell surface IL4R, with close blocking capacity to the positive controls.

### Example 5. Cell Based Functional Assay of Exemplary Bispecific Antibodies

### 5.1 Cell-Based Reporter Assay

A cell-based reporter assay was performed to evaluate the neutralizing activity of bispecific antibodies on TSLP-induced cellular STAT5-Luc reporter gene expression, using a reporter cell line HEK293T-TSLPR/IL7R/STAT5-Luc 5C5, as described in Example 4, that expressed cell-surface human TSLPR (SEQ ID NO: 36) and human IL7Ra (SEQ ID NO: 37).

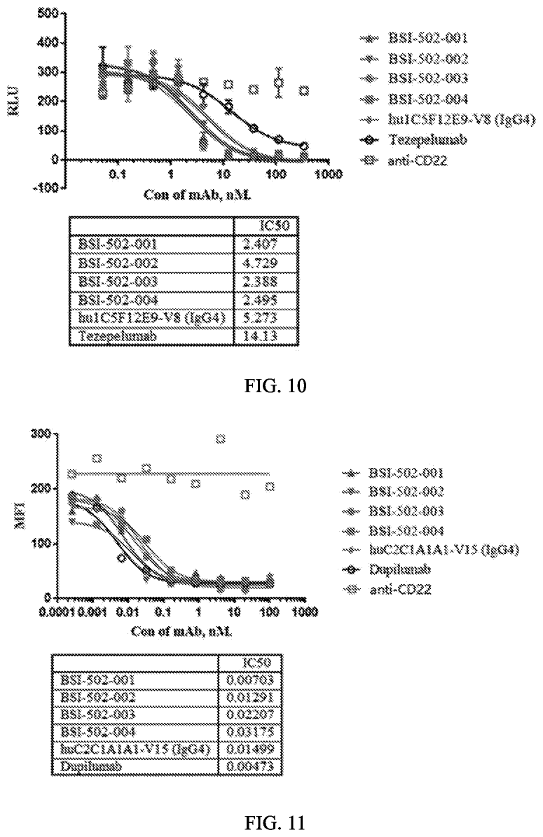

Briefly, the HEK293T-TSLPR/IL7R/STAT5-Luc 5C5 cells were harvested from cell culture flasks. Then, 5×10⁴ cells in 100 μL DMEM medium (Gibco, Cat #10566-016) supplemented with 10% FBS (Gibco, Cat #10099-141) were plated onto the 96 well cell culture plates. Meanwhile, 60 μL human TSLP-his (TSLP of SEQ ID NO: 32 with C-terminal His tag, 0.3 μg/mL in DMEM medium supplemented with 10% FBS) was respectively mixed with 60 μL serially diluted bispecific antibodies and controls, including an in house made anti-CD22 antibody, (3-fold dilution in DMEM medium supplemented with 10% FBS, starting at 333 nM), and the resultant mixtures were incubated for 30 minutes at room temperature. Then, the bispecific antibody/TSLP-his mixtures were added to the cell-coated plates, 100 μL/well, and the plates were incubated in a CO₂ incubator at 37° C. for 16 to 18 hours. Then 100 μL/well supernatants were discarded, and Luciferase detection Reagent (50 μL/well, Vazyme, Cat #DD1201-02) was added. Ten minutes later, the plates were subject to analysis by Tecan infinite 200Pro plate-reader. Luminescence signals were analyzed using Graphpad prism and IC₅₀ values were reported. The results were shown in FIG. 10.

The bispecific antibodies, including BSI-502-001, BSI-502-002, BSI-502-003 and BSI-502-004, effectively inhibited TSLP-induced STAT5 signaling pathway, while the isotype control anti-CD22 did not show any effect, indicating that the bispecific antibodies had specific inhibition activity. The bispecific antibodies' inhibitory activity was comparable to that of the parental anti-TSLP antibody hu1C5F12E9-V8(IgG4), and significantly better than that of Tezepelumab.

### 5.2 Cell-Based STAT6 Phosphorylation Assay

IL4 and IL13 have been reported to bind cell membrane human IL4Rα and induce phosphorylation of STAT6 in HEK293T-IL4R/STAT6/STAT6-Luc LB2 cells. STAT6 phosphorylation has been suggested to be critical for the IL4/IL13 signaling pathway.

The HEK293T-IL4R/STAT6/STAT6-Luc LB2 cells were prepared in house. Briefly, HEK293T cells (ATCC CRL-11268), naturally expressing IL13Rα1, were stably transfected with a pcDNA3.1-Puro (YouBio biological inc., Cat #VT9222) plasmid construct with the nucleotide encoding human IL4Rα inserted between BamHI and XhoI, a STAT6 plasmid (Sino biological inc., Cat #HG13190-NH) with the nucleotide encoding human STAT6 inserted between KpnI and XbaI, and a STAT6 Luciferase Reporter plasmid STAT6-Luc (Yeasen biological inc., Cat #11588ES03), and a single cell clone LB2 was chosen for subsequent functional assays.

Briefly, HEK293T-IL4R/STAT6/STAT6-Luc LB2 cells at the log phase stage were seeded into 96-well plates in 100 μL medium (RPMI1640+10% FBS), 2×10⁵ cells/well. Then, the plates were added with 50 μL serially diluted bispecific antibodies or controls (starting from 100 nM, 5-fold serial dilution), and incubated at 37° C. for 30 minutes. The plates were then added with 50 μL human IL4 protein (600 μg/mL, Sino biological inc., Cat #11846-HNAE) or human IL13 protein (60 ng/mL, Sino biological inc., Cat #10369-HNAC), and incubated at 37° C. for 20 minutes. The plates were centrifuged and washed twice using staining buffer (prepared in-house, DPBS+0.5% w/v BSA+2 mM EDTA), and then added with 50 μL/well fixation buffer (BD biosciences inc., Cat #5545655) and incubated for 30 minutes at 4° C. Cells were washed twice and incubated with permeabilization buffer (250 μl/well, BD biosciences inc., Cat #558050) for 30 minutes on ice. Plates were washed twice using staining buffer, added with anti-pSTAT6 antibody (50-fold dilution of Alexa Fluor®647 anti-STAT6 phospho Tyr641, Biolegend, Cat #686012) and left still for 60 minutes on ice. Plates were finally washed twice and resuspended in staining buffer. Fluorescence was measured using a Becton Dickinson FACS Canto II-HTS equipment. Data was analyzed using Graphpad Prism and IC₅₀ values were reported.

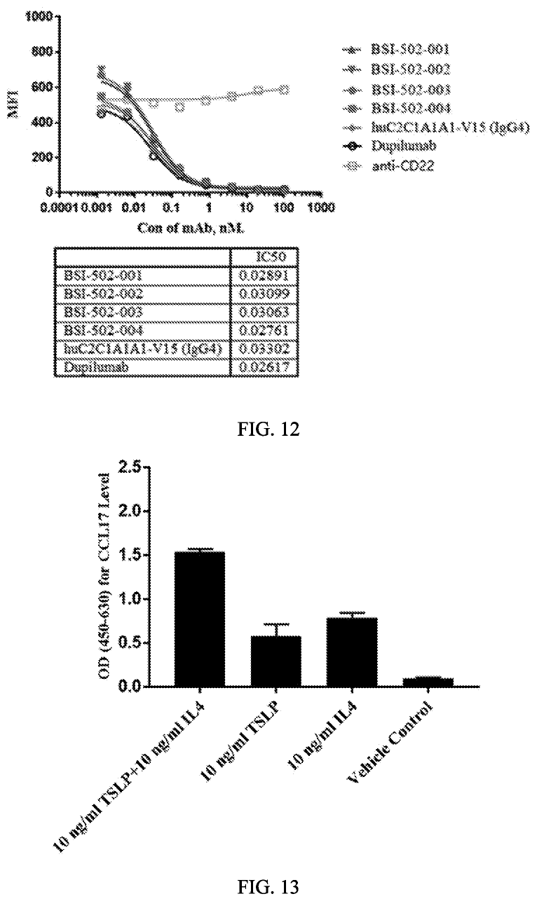

The results were shown in Table 3 and FIGS. 11 and 12.

TABLE 3
Bispecific antibodies' functional assay results

| | Inhibition on | Inhibition on |
|---|---|---|
| | IL4 induced | IL13 induced |
| | STAT6 | STAT6 |
| Bispecific | phosphorylation | phosphorylation |
| Ab ID# | (IC₅₀, nM) | (IC₅₀, nM) |
| BSI-502-001 | 0.00703 | 0.02891 |
| BSI-502-002 | 0.01291 | 0.03099 |
| BSI-502-003 | 0.02207 | 0.03063 |
| BSI-502-004 | 0.03175 | 0.02761 |
| huC2C1A1A1-V15(IgG4) | 0.01499 | 0.03302 |
| Dupilumab | 0.00473 | 0.02617 |

It can be seen that the bispecific antibodies of the disclosure were able to block IL4 or IL13-induced STAT6 phosphorylation in HEK293T-IL4R/STAT6/STAT6-Luc LB2 cells, while isotype control anti-CD22 did not show any effect, indicating that such inhibitory effect of the bispecific antibodies was specific. The bispecific antibodies' inhibitory activity was comparable to those of huC2C1A1A1-V15(IgG4) and Dupilumab.

### Example 6. Ex Vivo Inhibitory Effect of Exemplary Bispecific Antibodies on TSLP and/or IL4 Induced CCL-17 Production in Human PBMCs

6.1 TSLP and/or IL4 Induced CCL-17 Production in Human PBMCs Ex Vivo

CCL-17 (C-C motif chemokine ligand 17), also known as TARC (thymus and activation-regulated chemokine) is a member of the CC chemokines and plays an important role in allergic diseases such as atopic dermatitis and bronchial asthma. High serum concentrations of CCL-17 were observed in patients with atopic dermatitis, and closely related to disease activity (Umeda, M., et al. (2020) Sci Rep 10(1):6010).

The induction activity of TSLP and/or IL4 on CCL-17 production was evaluated in human PBMC (peripheral blood mononuclear cell) based bioassay. Briefly, human PBMCs (Milestone, P122011103C) were collected by centrifugation at 300 g for 8 minutes at room temperature, and resuspended in 3 mL RPMI1640 (Gibco, Cat #A10491-01) with 10% FBS. Then, 96-well plates were added with 120,000 cells per well in 100 μL RPMI1640 with 10% FBS, and incubated in a 5% C02 incubator at 37° C. for 2 hours. The plates were respectively added with i) human IL4 (Stemcell, Cat #78045.1) and human TSLP-his (TSLP of SEQ ID NO: 32 with C-terminal His tag) in RPMI1640 with 10% FBS, both with the final concentration of 10 ng/mL, ii) human TSLP-his in RPMI1640 with 10% FBS with the final concentration of 10 ng/mL, iii) human IL4 in RPMI1640 with 10% FBS with the final concentration of 10 ng/mL, and vi) RPMI1640 medium, 100 μL/well. After incubation in a 5% C02 incubator at 37° C. for 48 hours, the cell supernatants were collected and measured for CCL17 concentration using an ELISA Kit (Elabscience, Cat #E-EL-H0026C). The ELISA plates were detected in PERLONG #DNM-9602 Microplate reader for ODs (450-630). The data was analyzed using Graphpad Prism and the results were shown in FIG. 13.

According to FIG. 13, both the IL4 and TSLP alone can induce CCL17 production by PBMCs, and the IL4 and TSLP combination triggered CCL17 production at a much higher level.

### 6.2 Ex Vivo Effect of Exemplary Bispecific Antibodies on CCL-17 Production in Human PBMCs

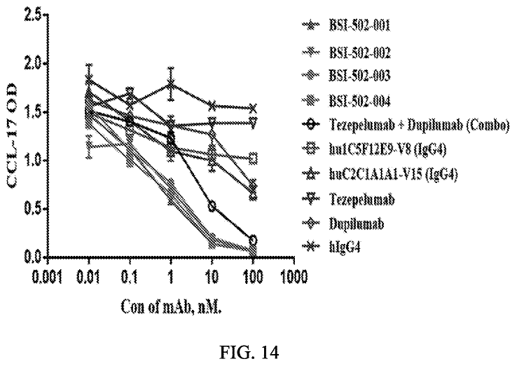

Following the protocol above, the human PBMCs were collected and incubated in 96-well plates in a 5% CO₂ incubator at 37° C. for 2 hours. The plates were added with 50 μL serially diluted bispecific antibodies of the disclosure, monospecific anti-TSLP mAbs, anti-IL4R mAbs, or the combination of Tezepelumab and Dupilumab, 10-fold dilution in RPMI1640 with 10% FBS starting from 100 nM (for the combination, starting at 100 nM Tezepelumab and 100 nM Dupilumab). The plates were then respectively added with 25 μL human TSLP-his and 25 μL human IL4 in RPMI1640 with 10% FBS, both at the final concentration of 10 ng/mL. After incubation in a 5% C02 incubator at 37° C. for 48 hours, the cell supernatants were collected and measured for CCL17 concentration using an ELISA Kit (Elabscience, Cat #E-EL-H0026C). The ELISA plates were detected in PERLONG #DNM-9602 Microplate reader for OD (450-630). Data was analyzed using Graphpad Prism and the results were shown in FIG. 14.

It can be seen that the bispecific antibodies of the disclosure effectively inhibited “TSLP+IL4” induced CCL-17 production by human PBMCs, at significantly higher activity than the monospecific antibodies alone or even the combination of the monospecific antibodies, indicating that the TSLP targeting may synergize with the IL4R targeting in inhibiting CCL-17 production.

While the disclosure has been described above in connection with one or more embodiments, it should be understood that the disclosure is not limited to those embodiments, and the description is intended to cover all alternatives, modifications, and equivalents, as may be included within the spirit and scope of the appended claims. All referenced cited herein are further incorporated by reference in their entirety.

Sequences in the present application are summarized below.

Description/
Sequence/SEQ ID NO.
VH CDR1 of hu1C5F12E9-V8
TYWMH (SEQ ID NO: 1)
VH CDR2 of hu1C5F12E9-V8
VIDPSDSDTTYNQKFKG (SEQ ID NO: 2)
VH CDR3 of hu1C5F12E9-V8
SLDGYYDY (SEQ ID NO: 3)
VL CDR1 of hu1C5F12E9-V8
RPTENIYSYLA (SEQ ID NO: 4)
VL CDR2 of hu1C5F12E9-V8
FARTLAE (SEQ ID NO: 5)
VL CDR3 of hu1C5F12E9-V8
QHHYGTPWT (SEQ ID NO: 6)
VH of hu1C5F12E9-V8
QVQLVQSGAEVKKPGASVKVSCKASGYTFTTYWMHWVRQAPGQGLEWMGVIDPSDSDTTY
NQKFKGRVTMTRDTSTSTVYMELSSLRSEDTAVYYCTRSLDGYYDYWGQGTLVTVSS (SEQ
ID NO: 7)
VL of hu1C5F12E9-V8
DIQMTQSPSSLSASVGDRVTITCRPTENIYSYLAWYQQKPGKAPKLLIYFARTLAEGVPSRFSG
SGSGTDFTLTISSLQPEDFATYYCQHHYGTPWTFGGGTKVEIK (SEQ ID NO: 8)
IgG1 heavy chain constant region
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQYNSTYRVVSVLTV
LHQDWLNGKEYKCQVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHN
HYTQKSLSLSPG (SEQ ID NO: 9)
Light chain constant region
RTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC (SEQ ID NO: 10)
VH CDR1 of huC2C1A1A1-V15
TYGMS (SEQ ID NO: 11)
VH CDR2 of huC2C1A1A1-V15
TINSNGGSTSYPDSVKG (SEQ ID NO: 12)
VH CDR3 of huC2C1A1A1-V15
FFRFRNAMDY (SEQ ID NO: 13)
VL CDR1 of huC2C1A1A1-V15
RTSENIYSYLA (SEQ ID NO: 14)
VL CDR2 of huC2C1A1A1-V15
NAKTLAE (SEQ ID NO: 15)
VL CDR3 of huC2C1A1A1-V15
QHYYGPPTWT (SEQ ID NO: 16)
VH of huC2C1A1A1-V15
EVQLVESGGGLVQPGGSLRLSCAASGFTFSTYGMSWVRQAPGKGLVWVATINSNGGSTSYPD
SVKGRFTISRDNAKNTLYLQMNSLRAEDTAVYYCARFFRFRNAMDYWGQGTLVTVSS (SEQ
ID NO: 17)
VL of huC2C1A1A1-V15
DIQMTQSPSSLSASVGDRVTITCRTSENIYSYLAWYQQKPGKAPKFLVYNAKTLAEGVPSRFS
GSGSGTDFTLTISSLQPEDFATYYCQHYYGPPTWTFGQGTKVEIK (SEQ ID NO: 18)
Linker
GGGGSGGGGS (SEQ ID NO: 19)
GGGGSGGGGSGGGGSGGGGS (SEQ ID NO: 20)
anti-IL4R heavy chain-linker-anti-TSLP V_{H}-V_{L}
EVQLVESGGGLVQPGGSLRLSCAASGFTFSTYGMSWVRQAPGKGLVWVATINSNGGSTSYPD
SVKGRFTISRDNAKNTLYLQMNSLRAEDTAVYYCARFFRFRNAMDYWGQGTLVTVSSASTK
GPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAQGGPSVFLFPPKPKD
TLYITREPEVTCVVVDVSHEDPEVKFNWYVDGVEVHNAKTKPREEQYNSTYRVVSVLTVLHQ
DWLNGKEYKCQVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYT
QKSLSLSPGKGGGGSGGGGSQVQLVQSGAEVKKPGASVKVSCKASGYTFTTYWMHWVRQAP
GQGLEWMGVIDPSDSDTTYNQKFKGRVTMTRDTSTSTVYMELSSLRSEDTAVYYCTRSLDG
YYDYWGQGTLVTVSSGGGGSGGGGSGGGGSGGGGSDIQMTQSPSSLSASVGDRVTITCRPTE
NIYSYLAWYQQKPGKAPKLLIYFARTLAEGVPSRFSGSGSGTDFTLTISSLQPEDFATYYCQHH
YGTPWTFGGGTKVEIK (SEQ ID NO: 21)
anti-IL4R heavy chain-linker-anti-TSLP V_{L}-V_{H}
EVQLVESGGGLVQPGGSLRLSCAASGFTFSTYGMSWVRQAPGKGLVWVATINSNGGSTSYPD
SVKGRFTISRDNAKNTLYLQMNSLRAEDTAVYYCARFFRFRNAMDYWGQGTLVTVSSASTK
GPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAQGGPSVFLFPPKPKD
TLYITREPEVTCVVVDVSHEDPEVKFNWYVDGVEVHNAKTKPREEQYNSTYRVVSVLTVLHQ
DWLNGKEYKCQVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYT
QKSLSLSPGKGGGGSGGGGSDIQMTQSPSSLSASVGDRVTITCRPTENIYSYLAWYQQKPGKA
PKLLIYFARTLAEGVPSRFSGSGSGTDFTLTISSLQPEDFATYYCQHHYGTPWTFGGGTKVEIK
GGGGSGGGGSGGGGSGGGGSQVQLVQSGAEVKKPGASVKVSCKASGYTFTTYWMHWVRQA
PGQGLEWMGVIDPSDSDTTYNQKFKGRVTMTRDTSTSTVYMELSSLRSEDTAVYYCTRSLDG
YYDYWGQGTLVTVSS (SEQ ID NO: 22)
anti-IL4R V_{H}-V_{L}-linker-anti-TSLP heavy chain
EVQLVESGGGLVQPGGSLRLSCAASGFTFSTYGMSWVRQAPGKGLVWVATINSNGGSTSYPD
SVKGRFTISRDNAKNTLYLQMNSLRAEDTAVYYCARFFRFRNAMDYWGQGTLVTVSSGGGG
SGGGGSGGGGSGGGGSDIQMTQSPSSLSASVGDRVTITCRTSENIYSYLAWYQQKPGKAPKFL
VYNAKTLAEGVPSRFSGSGSGTDFTLTISSLQPEDFATYYCQHYYGPPTWTFGQGTKVEIKGG
GGSGGGGSQVQLVQSGAEVKKPGASVKVSCKASGYTFTTYWMHWVRQAPGQGLEWMGVID
PSDSDTTYNQKFKGRVTMTRDTSTSTVYMELSSLRSEDTAVYYCTRSLDGYYDYWGQGTLV
TVSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAQGGPSVF
LFPPKPKDTLYITREPEVTCVVVDVSHEDPEVKFNWYVDGVEVHNAKTKPREEQYNSTYRVVS
VLTVLHQDWLNGKEYKCQVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHEA
LHNHYTQKSLSLSPG (SEQ ID NO: 23)
anti-IL4R V_{L}-V_{H}-linker-anti-TSLP heavy chain
DIQMTQSPSSLSASVGDRVTITCRTSENIYSYLAWYQQKPGKAPKFLVYNAKTLAEGVPSRFS
GSGSGTDFTLTISSLQPEDFATYYCQHYYGPPTWTFGQGTKVEIKGGGGSGGGGSGGGGSGG
GGSEVQLVESGGGLVQPGGSLRLSCAASGFTFSTYGMSWVRQAPGKGLVWVATINSNGGSTS
YPDSVKGRFTISRDNAKNTLYLQMNSLRAEDTAVYYCARFFRFRNAMDYWGQGTLVTVSSG
GGGSGGGGSQVQLVQSGAEVKKPGASVKVSCKASGYTFTTYWMHWVRQAPGQGLEWMGVI
DPSDSDTTYNQKFKGRVTMTRDTSTSTVYMELSSLRSEDTAVYYCTRSLDGYYDYWGQGTL
VTVSSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAQGGPSV
FLFPPKPKDTLYITREPEVTCVVVDVSHEDPEVKFNWYVDGVEVHNAKTKPREEQYNSTYRVV
SVLTVLHQDWLNGKEYKCQVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMHE
ALHNHYTQKSLSLSPG (SEQ ID NO: 24)
anti-TSLP heavy chain-linker-anti-IL4R V_{H}-V_{L}
QVQLVQSGAEVKKPGASVKVSCKASGYTFTTYWMHWVRQAPGQGLEWMGVIDPSDSDTTY
NQKFKGRVTMTRDTSTSTVYMELSSLRSEDTAVYYCTRSLDGYYDYWGQGTLVTVSSASTK
GPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAQGGPSVFLFPPKPKD
TLYITREPEVTCVVVDVSHEDPEVKFNWYVDGVEVHNAKTKPREEQYNSTYRVVSVLTVLHQ
DWLNGKEYKCQVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYT
QKSLSLSPGKGGGGSGGGGSEVQLVESGGGLVQPGGSLRLSCAASGFTFSTYGMSWVRQAPG
KGLVWVATINSNGGSTSYPDSVKGRFTISRDNAKNTLYLQMNSLRAEDTAVYYCARFFRFRN
AMDYWGQGTLVTVSSGGGGSGGGGSGGGGSGGGGSDIQMTQSPSSLSASVGDRVTITCRTSE
NIYSYLAWYQQKPGKAPKFLVYNAKTLAEGVPSRFSGSGSGTDFTLTISSLQPEDFATYYCQH
YYGPPTWTFGQGTKVEIK (SEQ ID NO: 25)
anti-TSLP heavy chain-linker-anti-IL4R V_{L}-V_{H}
QVQLVQSGAEVKKPGASVKVSCKASGYTFTTYWMHWVRQAPGQGLEWMGVIDPSDSDTTY
NQKFKGRVTMTRDTSTSTVYMELSSLRSEDTAVYYCTRSLDGYYDYWGQGTLVTVSSASTK
GPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAQGGPSVFLFPPKPKD
TLYITREPEVTCVVVDVSHEDPEVKFNWYVDGVEVHNAKTKPREEQYNSTYRVVSVLTVLHQ
DWLNGKEYKCQVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYT
QKSLSLSPGKGGGGSGGGGSDIQMTQSPSSLSASVGDRVTITCRTSENIYSYLAWYQQKPGKAP
KFLVYNAKTLAEGVPSRFSGSGSGTDFTLTISSLQPEDFATYYCQHYYGPPTWTFGQGTKVEIK
GGGGSGGGGSGGGGSGGGGSEVQLVESGGGLVQPGGSLRLSCAASGFTFSTYGMSWVRQAP
GKGLVWVATINSNGGSTSYPDSVKGRFTISRDNAKNTLYLQMNSLRAEDTAVYYCARFFRFR
NAMDYWGQGTLVTVSS (SEQ ID NO: 26)
anti-TSLP V_{H}-V_{L}-linker-anti-ILAR heavy chain
QVQLVQSGAEVKKPGASVKVSCKASGYTFTTYWMHWVRQAPGQGLEWMGVIDPSDSDTTY
NQKFKGRVTMTRDTSTSTVYMELSSLRSEDTAVYYCTRSLDGYYDYWGQGTLVTVSSGGGG
SGGGGSGGGGSGGGGSDIQMTQSPSSLSASVGDRVTITCRPTENIYSYLAWYQQKPGKAPKLLI
YFARTLAEGVPSRFSGSGSGTDFTLTISSLQPEDFATYYCQHHYGTPWTFGGGTKVEIKGGGG
SGGGGSEVQLVESGGGLVQPGGSLRLSCAASGFTFSTYGMSWVRQAPGKGLVWVATINSNG
GSTSYPDSVKGRFTISRDNAKNTLYLQMNSLRAEDTAVYYCARFFRFRNAMDYWGQGTLVT
VSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPEAQGGPSVFLF
PPKPKDTLYITREPEVTCVVVDVSHEDPEVKFNWYVDGVEVHNAKTKPREEQYNSTYRVVSVL
TVLHQDWLNGKEYKCQVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEAL
HNHYTQKSLSLSPG (SEQ ID NO: 27)
anti-TSLP V_{L}-V_{H}-linker-anti-IL4R heavy chain
DIQMTQSPSSLSASVGDRVTITCRPTENIYSYLAWYQQKPGKAPKLLIYFARTLAEGVPSRFSG
SGSGTDFTLTISSLQPEDFATYYCQHHYGTPWTFGGGTKVEIKGGGGSGGGGSGGGGSGGGG
SQVQLVQSGAEVKKPGASVKVSCKASGYTFTTYWMHWVRQAPGQGLEWMGVIDPSDSDTT
YNQKFKGRVTMTRDTSTSTVYMELSSLRSEDTAVYYCTRSLDGYYDYWGQGTLVTVSSGGG
GSGGGGSEVQLVESGGGLVQPGGSLRLSCAASGFTFSTYGMSWVRQAPGKGLVWVATINSNG
GSTSYPDSVKGRFTISRDNAKNTLYLQMNSLRAEDTAVYYCARFFRFRNAMDYWGQGTLVT
VSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPEAQGGPSVFLF
PPKPKDTLYITREPEVTCVVVDVSHEDPEVKFNWYVDGVEVHNAKTKPREEQYNSTYRVVSVL
TVLHQDWLNGKEYKCQVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEAL
HNHYTQKSLSLSPG (SEQ ID NO: 28)
IgG4 heavy chain constant region
ASTKGPSVFPLAPCSRSTSESTAALGCLVKDYFPEPVTVSWNSGALTSGVHTFPAVLQSSGLYSL
SSVVTVPSSSLGTKTYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQ
KSLSLSLGK (SEQ ID NO: 29)
Heavy chain of Tezepelumab
QMQLVESGGGVVQPGRSLRLSCAASGFTFRTYGMHWVRQAPGKGLEWVAVIWYDGSNKHY
ADSVKGRFTITRDNSKNTLNLQMNSLRAEDTAVYYCARAPQWELVHEAFDIWGQGTMVTVSS
ASTKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAGPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPSD
IAVEWESNGQPENNYKTTPPMLDSDGSFFLYSKLTVDKSRWQQGNVFSCSVMHEALHNHYTQ
KSLSLSPGK (SEQ ID NO: 30)
Light chain of Tezepelumab
SYVLTQPPSVSVAPGQTARITCGGNNLGSKSVHWYQQKPGQAPVLVVYDDSDRPSWIPERFSG
SNSGNTATLTISRGEAGDEADYYCQVWDSSSDHVVFGGGTKLTVLGQPKAAPSVTLFPPSSEEL
QANKATLVCLISDFYPGAVTVAWKADSSPVKAGVETTTPSKQSNNKYAASSYLSLTPEQWKSH
RSYSCQVTHEGSTVEKTVAPTECS (SEQ ID NO: 31)
human TSLP
YDFTNCDFEKIKAAYLSTISKDLITYMSGTKSTEFNNTVSCSNRPHCLTEIQSLTFNPTAGCASLA
KEMFAMKTKAALAIWCPGYSETQINATQAMKKRRKRKVTTNKCLEQVSQLQGLWRRFNRPL
LKQQ (SEQ ID NO: 32)
human IL4Rα
MGWLCSGLLFPVSCLVLLQVASSGNMKVLQEPTCVSDYMSISTCEWKMNGPTNCSTELRLLY
QLVFLLSEAHTCIPENNGGAGCVCHLLMDDVVSADNYTLDLWAGQQLLWKGSFKPSEHVKPR
APGNLTVHTNVSDTLLLTWSNPYPPDNYLYNHLTYAVNIWSENDPADFRIYNVTYLEPSLRIAA
STLKSGISYRARVRAWAQCYNTTWSEWSPSTKWHNSYREPFEQ (SEQ ID NO: 33)
human TSLPR
GAAEGVQIQIIYFNLETVQVTWNASKYSRTNLTFHYRFNGDEAYDQCTNYLLQEGHTSGCLLD
AEQRDDILYFSIRNGTHPVFTASRWMVYYLKPSSPKHVRFSWHQDAVTVTCSDLSYGDLLYEV
QYRSPFDTEWQSKQENTCNVTIEGLDAEKCYSFWVRVKAMEDVYGPDTYPSDWSEVTCWQR
GEIRDACAETPTPPKPKLSKDIEGRMD (SEQ ID NO: 34)
human IL7Ra protein
ESGYAQNGDLEDAELDDYSFSCYSQLEVNGSQHSLTCAFEDPDVNTTNLEFEICGALVEVKCL
NFRKLQEIYFIETKKFLLIGKSNICVKVGEKSLTCKKIDLTTIVKPEAPFDLSVIYREGANDFVVTF
NTSHLQKKYVKVLMHDVAYRQEKDENKWTHVNLSSTKLTLLQRKLQPAAMYEIKVRSIPDHY
FKGFWSEWSPSYYFRTPEINNSSG (SEQ ID NO: 35)
human TSLPR
MGRLVLLWGAAVFLLGGWMALGQGGAAEGVQIQIIYFNLETVQVTWNASKYSRTNLTFHYRF
NGDEAYDQCTNYLLQEGHTSGCLLDAEQRDDILYFSIRNGTHPVFTASRWMVYYLKPSSPKHV
RFSWHQDAVTVTCSDLSYGDLLYEVQYRSPFDTEWQSKQENTCNVTIEGLDAEKCYSFWVRV
KAMEDVYGPDTYPSDWSEVTCWQRGEIRDACAETPTPPKPKLSKFILISSLAILLMVSLLLLSLW
KLWRVKKFLIPSVPDPKSIFPGLFEIHQGNFQEWITDTQNVAHLHKMAGAEQESGPEEPLVVQL
AKTEAESPRMLDPQTEEKEASGGSLQLPHQPLQGGDVVTIGGFTFVMNDRSYVAL (SEQ ID NO:
36)
human IL7Ra
MTILGTTFGMVFSLLQVVSGESGYAQNGDLEDAELDDYSFSCYSQLEVNGSQHSLTCAFEDPD
VNTTNLEFEICGALVEVKCLNFRKLQEIYFIETKKFLLIGKSNICVKVGEKSLTCKKIDLTTIVKPE
APFDLSVIYREGANDFVVTFNTSHLQKKYVKVLMHDVAYRQEKDENKWTHVNLSSTKLTLLQ
RKLQPAAMYEIKVRSIPDHYFKGFWSEWSPSYYFRTPEINNSSGEMDPILLTISILSFFSVALLVIL
ACVLWKKRIKPIVWPSLPDHKKTLEHLCKKPRKNLNVSFNPESFLDCQIHRVDDIQARDEVEGF
LQDTFPQQLEESEKQRLGGDVQSPNCPSEDVVVTPESFGRDSSLTCLAGNVSACDAPILSSSRSL
DCRESGKNGPHVYQDLLLSLGTTNSTLPPPFSLQSGILTLNPVAQGQPILTSLGSNQEEAYVTMS
SFYQNQ (SEQ ID NO: 37)
human IgG-Fc
EPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPGK (SEQ ID NO: 38)
anti-IL4R light chain variable region-light chain constant region
SEQ ID NO: 39, comprising from N- to C-terminus SEQ ID NO: 18 and SEQ ID NO: 10
anti-TSLP light chain variable region-light chain constant region
SEQ ID NO: 40, comprising from N- to C-terminus SEQ ID NO: 8 and SEQ ID NO: 10

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A recombinant antibody, comprising:
i) an anti-TSLP antibody or an antigen binding portion thereof, and
ii) an anti-IL4R single-chain variable region (scFv); or
i) an anti-IL4R antibody or an antigen binding portion thereof, and
ii) an anti-TSLP single-chain variable region (scFv).

2. A recombinant antibody of claim 1, comprising:
i) an antagonistic anti-TSLP antibody, comprising an anti-TSLP heavy chain variable region, a heavy chain constant region, an anti-TSLP light chain variable region, and an light chain constant region, and
ii) an antagonistic anti-IL4R single-chain variable region (scFv), comprising an anti-IL4R heavy chain variable region and an anti-IL4R light chain variable region; or
i) an antagonistic anti-IL4R antibody, comprising an anti-IL4R heavy chain variable region, a heavy chain constant region, an anti-IL4R light chain variable region, and an light chain constant region, and
ii) an antagonistic anti-TSLP single-chain variable region (scFv), comprising an anti-TSLP heavy chain variable region and an anti-TSLP light chain variable region.

3. The recombinant antibody of claim 2, wherein,
the anti-IL4R scFv is linked to the N-terminus of the anti-TSLP heavy chain variable region, the N-terminus of the anti-TSLP light chain variable region, the C-terminus of the anti-TSLP heavy chain constant region, or the C-terminus of the anti-TSLP light chain constant region; or
the anti-TSLP scFv is linked to the N-terminus of the anti-IL4R heavy chain variable region, the N-terminus of the anti-IL4R light chain variable region, the C-terminus of the anti-IL4R heavy chain constant region, or the C-terminus of the anti-IL4R light chain constant region.

4. The recombinant antibody of claim 2, wherein the anti-TSLP heavy chain variable region comprises a VH CDR1, a VH CDR2 and a VH CDR3 comprising the amino acid sequences of SEQ ID NOs: 1, 2 and 3, respectively, and the anti-TSLP light chain variable region comprises a VL CDR1, a VL CDR2 and a VL CDR3 comprising the amino acid sequences of SEQ ID NOs: 4, 5 and 6, respectively.

5. The recombinant antibody of claim 4, wherein the anti-TSLP heavy chain variable region and the anti-TSLP light chain variable region comprise amino acid sequences having at least 85%, 86%, 87%, 88%, 89%, 90%, 91%, 92%, 93%, 94%, 95%, 96%, 97%, 98%, 99% or 100% identity to SEQ ID NOs: 7 and 8, respectively.

6. The recombinant antibody of claim 2, wherein the anti-IL4R heavy chain variable region comprises a VH CDR1, a VH CDR2 and a VH CDR3 comprising the amino acid sequences of SEQ ID NOs: 11, 12 and 13, respectively, and the anti-IL4R light chain variable region comprises a VL CDR1, a VL CDR2 and a VL CDR3 comprising the amino acid sequences of SEQ ID NOs: 14, 15 and 16, respectively.

7. The recombinant antibody of claim 6, wherein the anti-IL4R heavy chain variable region and the anti-IL4R light chain variable region comprise amino acid sequences having at least 85%, 86%, 87%, 88%, 89%, 90%, 91%, 92%, 93%, 94%, 95%, 96%, 97%, 98%, 99% or 100% identity to SEQ ID NOs: 17 and 18, respectively.

8. The recombinant antibody of claim 2, wherein heavy chain constant region is human IgG1, IgG2 or IgG4 constant region.

9. The recombinant antibody of claim 2, comprising
i) a first polypeptide chain and a second polypeptide chain each comprising, from N- to C-terminus, the anti-IL4R scFv, an optional linker, the anti-TSLP heavy chain variable region, and the heavy chain constant region, and
a third polypeptide chain and a fourth polypeptide chain each comprising, from N- to C-terminus, the anti-TSLP light chain variable region and the light chain constant region;
ii) a first polypeptide chain and a second polypeptide chain each comprising, from N- to C-terminus, the anti-TSLP heavy chain variable region and the heavy chain constant region, and
a third polypeptide chain and a fourth polypeptide chain each comprising, from N- to C-terminus, the anti-IL4R scFv, an optional linker, the anti-TSLP light chain variable region and the light chain constant region;
iii) a first polypeptide chain and a second polypeptide chain each comprising, from N- to C-terminus, the anti-TSLP heavy chain variable region, the heavy chain constant region, an optional linker, and the anti-IL4R scFv, and
a third polypeptide chain and a fourth polypeptide chain each comprising, from N- to C-terminus, the anti-TSLP light chain variable region and the light chain constant region;
iv) a first polypeptide chain and a second polypeptide chain each comprising, from N- to C-terminus, the anti-TSLP scFv, an optional linker, the anti-IL4R heavy chain variable region, and the heavy chain constant region, and
a third polypeptide chain and a fourth polypeptide chain each comprising, from N- to C-terminus, the anti-IL4R light chain variable region and the light chain constant region;
v) a first polypeptide chain and a second polypeptide chain each comprising, from N- to C-terminus, the anti-IL4R heavy chain variable region and the heavy chain constant region, and
a third polypeptide chain and a fourth polypeptide chain each comprising, from N- to C-terminus, the anti-TSLP scFv, an optional linker, the anti-IL4R light chain variable region and the light chain constant region; or
vi) a first polypeptide chain and a second polypeptide chain each comprising, from N- to C-terminus, the anti-IL4R heavy chain variable region, the heavy chain constant region, an optional linker, and the anti-TSLP scFv, and
a third polypeptide chain and a fourth polypeptide chain each comprising, from N- to C-terminus, the anti-IL4R light chain variable region and the light chain constant region,
wherein the anti-IL4R heavy chain variable region in the first polypeptide chain and the anti-IL4R light chain variable region in the third polypeptide chain associate to form an IL4R binding domain, the anti-IL4R heavy chain variable region in the second polypeptide chain and the anti-IL4R light chain variable region in the fourth polypeptide chain associate to form an IL4R binding domain,
wherein the anti-TSLP heavy chain variable region in the first polypeptide chain and the anti-TSLP light chain variable region in the third polypeptide chain associate to form a TSLP binding domain, the anti-TSLP heavy chain variable region in the second polypeptide chain and the anti-TSLP light chain variable region in the fourth polypeptide chain associate to form a TSLP binding domain,
wherein the anti-IL4R scFv comprises from N- to C-terminus the anti-IL4R heavy chain variable region, an optional linker and the anti-IL4R light chain variable region, or the anti-IL4R light chain variable region, an optional linker, and the anti-IL4R heavy chain variable region,
wherein the anti-TSLP scFv comprises from N- to C-terminus the anti-TSLP heavy chain variable region, an optional linker and the anti-TSLP light chain variable region, or the anti-TSLP light chain variable region, an optional linker, and the anti-TSLP heavy chain variable region,
wherein the heavy chain constant region in the first polypeptide chain and the heavy chain constant region in the second polypeptide chain are associated together.

10. The recombinant antibody of claim 9, wherein the first polypeptide chain, the second polypeptide chain, the third polypeptide chain, and the fourth polypeptide chain comprise amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% identity to
i) SEQ ID NOs: 21, 21, 39 and 39, respectively;
ii) SEQ ID NOs: 22, 22, 39 and 39, respectively;
iii) SEQ ID NOs: 23, 23, 40 and 40, respectively;
iv) SEQ ID NOs: 24, 24, 40 and 40, respectively;
v) SEQ ID NOs: 25, 25, 40 and 40, respectively;
vi) SEQ ID NOs: 26, 26, 40 and 40, respectively;
vii) SEQ ID NOs: 27, 27, 39 and 39, respectively; or
viii) SEQ ID NOs: 28, 28, 39 and 39, respectively.

11. A nucleic acid molecule, encoding the recombinant antibody of claim 1 or 2.

12. An expression vector, comprising the nucleic acid molecule of claim 11.

13. A host cell, comprising the expression vector of claim 12 or having the nucleic acid molecule of claim 11 integrated into its genome.

14. A pharmaceutical composition, comprising the recombinant antibody of claim 1 or 2, and a pharmaceutically acceptable carrier.

15. A method for treating a disease associated with TSLP or IL4/IL13 in a subject in need thereof, comprising administering to the subject the pharmaceutical composition of claim 14.

16. The method of claim 15, wherein the disease is an inflammatory disease.

17. The method of claim 16, wherein the disease is atopic dermatitis, asthma, chronic rhinosinusitis, nasal polyposis, or eosinophilic esophagitis.

18. A kit comprising the recombinant antibody of claim 1 or 2.


SEQUENCE LISTING (40 sequences)

SEQ ID NO: 1 - protein, 5 aa, synthetic construct
TYWMH

SEQ ID NO: 2 - protein, 17 aa, synthetic construct
VIDPSDSDTTYNQKFKG

SEQ ID NO: 3 - protein, 8 aa, synthetic construct
SLDGYYDY

SEQ ID NO: 4 - protein, 11 aa, synthetic construct
RPTENIYSYLA

SEQ ID NO: 5 - protein, 7 aa, synthetic construct
FARTLAE

SEQ ID NO: 6 - protein, 9 aa, synthetic construct
QHHYGTPWT

SEQ ID NO: 7 - protein, 117 aa, synthetic construct
QVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSS

SEQ ID NO: 8 - protein, 107 aa, synthetic construct
DIQMTQSPSSLSASVGDRVTITCRPTENIYSYLAWYQQKPGKAPKLLIYFARTLAEGVPSRFSGSGSGTDFTLTISSLQPEDFATYYCQHHYGTPWTFGGGTKVEIK

SEQ ID NO: 9 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 107 aa, synthetic construct [sequence omitted]

SEQ ID NO: 11 - protein, 5 aa, synthetic construct
TYGMS

SEQ ID NO: 12 - protein, 17 aa, synthetic construct
TINSNGGSTSYPDSVKG

SEQ ID NO: 13 - protein, 10 aa, synthetic construct
FFRFRNAMDY

SEQ ID NO: 14 - protein, 11 aa, synthetic construct
RTSENIYSYLA

SEQ ID NO: 15 - protein, 7 aa, synthetic construct
NAKTLAE

SEQ ID NO: 16 - protein, 10 aa, synthetic construct
QHYYGPPTWT

SEQ ID NO: 17 - protein, 119 aa, synthetic construct
EVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSS

SEQ ID NO: 18 - protein, 108 aa, synthetic construct
DIQMTQSPSSLSASVGDRVTITCRTSENIYSYLAWYQQKPGKAPKFLVYNAKTLAEGVPSRFSGSGSGTDFTLTISSLQPEDFATYYCQHYYGPPTWTFGQGTKVEIK

SEQ ID NO: 19 - protein, 10 aa, synthetic construct
GGGGSGGGGS

SEQ ID NO: 20 - protein, 20 aa, synthetic construct
GGGGSGGGGSGGGGSGGGGS

SEQ ID NO: 21 - protein, 703 aa, synthetic construct
EVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSSGGGGSGGGGSGGGGSGGGGSDIQMTQSPSSLSASVGDRVTITCRPTENIYSYLAWYQQKPGKAPKLLIYFARTLAEGVPSRFSGSGSGTDFTLTISSLQPEDFATYYCQHHYGTPWTFGGGTKVEIK

SEQ ID NO: 22 - protein, 703 aa, synthetic construct
EVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRPTENIYSYLAWYQQKPGKAPKLLIYFARTLAEGVPSRFSGSGSGTDFTLTISSLQPEDFATYYCQHHYGTPWTFGGGTKVEIKGGGGSGGGGSGGGGSGGGGSQVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSS

SEQ ID NO: 23 - protein, 703 aa, synthetic construct
EVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSSGGGGSGGGGSGGGGSGGGGSDIQMTQSPSSLSASVGDRVTITCRTSENIYSYLAWYQQKPGKAPKFLVYNAKTLAEGVPSRFSGSGSGTDFTLTISSLQPEDFATYYCQHYYGPPTWTFGQGTKVEIKGGGGSGGGGSQVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 703 aa, synthetic construct
DIQMTQSPSSLSASVGDRVTITCRTSENIYSYLAWYQQKPGKAPKFLVYNAKTLAEGVPSRFSGSGSGTDFTLTISSLQPEDFATYYCQHYYGPPTWTFGQGTKVEIKGGGGSGGGGSGGGGSGGGGSEVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSSGGGGSGGGGSQVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 704 aa, synthetic construct
QVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSSGGGGSGGGGSGGGGSGGGGSDIQMTQSPSSLSASVGDRVTITCRTSENIYSYLAWYQQKPGKAPKFLVYNAKTLAEGVPSRFSGSGSGTDFTLTISSLQPEDFATYYCQHYYGPPTWTFGQGTKVEIK

SEQ ID NO: 26 - protein, 704 aa, synthetic construct
QVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRTSENIYSYLAWYQQKPGKAPKFLVYNAKTLAEGVPSRFSGSGSGTDFTLTISSLQPEDFATYYCQHYYGPPTWTFGQGTKVEIKGGGGSGGGGSGGGGSGGGGSEVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSS

SEQ ID NO: 27 - protein, 702 aa, synthetic construct
QVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSSGGGGSGGGGSGGGGSGGGGSDIQMTQSPSSLSASVGDRVTITCRPTENIYSYLAWYQQKPGKAPKLLIYFARTLAEGVPSRFSGSGSGTDFTLTISSLQPEDFATYYCQHHYGTPWTFGGGTKVEIKGGGGSGGGGSEVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 702 aa, synthetic construct
DIQMTQSPSSLSASVGDRVTITCRPTENIYSYLAWYQQKPGKAPKLLIYFARTLAEGVPSRFSGSGSGTDFTLTISSLQPEDFATYYCQHHYGTPWTFGGGTKVEIKGGGGSGGGGSGGGGSGGGGSQVQLVQSGAEVKKPGASVKVSCKASGYTFTTYWMHWVRQAPGQGLEWMGVIDPSDSDTTYNQKFKGRVTMTRDTSTSTVYMELSSLRSEDTAVYYCTRSLDGYYDYWGQGTLVTVSSGGGGSGGGGSEVQLVESGGGLVQPGGSLRLSCAASGFTFSTYGMSWVRQAPGKGLVWVATINSNGGSTSYPDSVKGRFTISRDNAKNTLYLQMNSLRAEDTAVYYCARFFRFRNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 448 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 214 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 131 aa, Homo sapiens
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 33 - protein, 231 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQ

SEQ ID NO: 34 - protein, 214 aa, Homo sapiens
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKDIEGRMD

SEQ ID NO: 35 - protein, 216 aa, Homo sapiens
ESGYAQNGDLEDAELDDYSFSCYSQLEVNGSQHSLTCAFEDPDVNTTNLEFEICGALVEVKCLNFRKLQEIYFIETKKFLLIGKSNICVKVGEKSLTCKKIDLTTIVKPEAPFDLSVIYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSG

SEQ ID NO: 36 - protein, 371 aa, Homo sapiens
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 37 - protein, 459 aa, Homo sapiens [sequence omitted]

SEQ ID NO: 38 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 215 aa, synthetic construct
DIQMTQSPSSLSASVGDRVTITCRTSENIYSYLAWYQQKPGKAPKFLVYNAKTLAEGVPSRFSGSGSGTDFTLTISSLQPEDFATYYCQHYYGPPTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 214 aa, synthetic construct
DIQMTQSPSSLSASVGDRVTITCRPTENIYSYLAWYQQKPGKAPKLLIYFARTLAEGVPSRFSGSGSGTDFTLTISSLQPEDFATYYCQHHYGTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC